(19) United States
(12) Patent Application Publication
(10) Pub. No.: US 2021/0369698 A1
(43) Pub. Date: Dec. 2, 2021

(54) Treating Dermatological Conditions with Chloroquine and/or Hydroxychloroquine

(71) Applicants: Frank Anthony Spallitta, (US); Attillaps Holdings

(72) Inventor: Frank Anthony SPALLITTA, Denver, CO (US)

(21) Appl. No.: 16/959,936

(22) PCT Filed: Jan. 4, 2019
(86) PCT No.: PCT/US2019/012315

§ 371 Date: Jul. 2, 2020

(51) Int. Cl. A61K 31/4706; A61K 9/00; A61P 33/14

(52) U.S. Cl. CPC A61K 31/4706; A61P 33/14; A61K 9/0014

(57) ABSTRACT

Embodiments of the invention involve treating skin afflictions by the topical or oral use of chloroquine and/or hydroxychloroquines. By effectively reducing or eliminating the population of Demodex mites in affected skin areas and areas where Demodex mites may exist, this treatment achieves a more complete remission of clinical signs and symptoms of the skin afflictions than any previously described method. The invention is useful for treating a range of skin afflictions including common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis, rosacea, ocular rosacea/dry eye, and for diagnostic methods thereof.

### CROSS-REFERENCE TO RELATED APPLICATIONS

This application claims the benefit of priority to U.S. Provisional Patent Application Nos. 62/614,082 filed on Jan. 5, 2018, which is specifically incorporated by reference herein to the extent not inconsistent herewith.

### BACKGROUND

Provided herein are methods for treatment of various skin afflictions in humans employing topically applied, orally dosed, or intravenously introduced chloroquine and/or hydroxychloroquine. By reducing or eliminating Demodex organisms from affected skin areas, this method reduces clinical signs of the skin afflictions which are primarily due to allergic and vasomotor responses of the body to the organism and bacteria that are carried by the organism.

Rosacea, originally termed acne rosacea, is a chronic inflammatory skin condition most commonly affecting the face and eyelids of middle-aged adults. Clinical signs include erythema (redness), dryness, papules, pustules, and nodules either singly or in combination in the involved skin areas. Eyelid involvement may be manifested by mild conjunctival irritation or inflammation of the meibomian (oil) glands on the eyelid margin. Chronic eyelid irritation can result in loss of eyelashes. No visual impairment accompanies the eyelid irritation. Chronic involvement of the nose with rosacea in men can cause a bulbous enlargement known as rhinophyma. In the classic situation, the condition develops in adults between the ages of 30 and 50. While certain lesions of rosacea may mimic lesions of acne vulgaris, the processes are separate and distinct, the principal differences being the presence of comedones (whiteheads and blackheads) only in acne vulgaris and not in rosacea, the characteristic midfacial localization and flushing of rosacea not seen in acne, and the potential for eyelid involvement in rosacea which never occurs in acne. In fact, the clinical observation has been made that persons who have classic acne vulgaris as teenagers rarely, if ever, develop full-blown rosacea as adults.

Rosacea develops in four stages over several years, in spasms aggravated by variations in various conditions such as temperature, alcohol, spices, exposure to sunlight and emotions. The various disease stages can be described in terms of the stages: Stage 1: stage of erythema episodes. The patients have erythrosis spasms due to the sudden dilation of the arterioles of the face, which then take on a congestive, red appearance. These spasms are caused by the emotions, meals and temperature changes; Stage 2: stage of couperosis, i.e., of permanent erythema with telangiectasia. Certain patients also have edema on the cheeks and the forehead; Stage 3: inflammatory stage with appearance of inflammatory papules and pustules, but without affecting the sebaceous follicles and thus with absence of cysts and comedones; and Stage 4: rhinophyma stage. This late phase essentially affects men. The patients have a bumpy, voluminous red nose with sebaceous hyperplasia and fibrous reordering of the connective tissue.

Conventionally, rosacea is treated orally or topically with antibiotics such as tetracyclines, erythromycin or clindamycin, but also with vitamin A, salicylic acid, antifungal agents, steroids, anti-infectious agents such as benzoyl peroxide, or with isotretinoin in severe cases or most commonly with metronidazole (an antibacterial agent).

Metronidazole is known for its antiparasitic, antiprotozoan and antibacterial properties. It is especially used for treating Helicobacter pylori infections. It is also prescribed in the treatment of rosacea, for its advantageous properties on the inflammatory lesions of rosacea, specifically on papules and pustules. Metronidazole exerts selective toxicity towards anaerobic microorganisms and also hypoxic cells. On the latter, metronidazole is reduced to various derivatives that are capable of changing the structure of their DNA.

U.S. Patent Application 2013/0095051 filed Dec. 6, 2012 describes a method of treating rosacea using avermectin/metronidazole in a topical application. U.S. Pat. No. 5,952,372 describes a method for treating rosacea using ivermectin orally or topically in order to reduce and eliminate the parasite Demodex folliculorum present on the skin of patients.

Ivermectin belongs to the avermectin family, a group of macrocyclic lactones produced by the bacterium Streptomyces avermitilis. The avermectins especially include ivermectin, invermectin, avermectin, abamectin, doramectin, eprinomectin and selamectin. Ivermectin is known for its antiparasitic and anthelmintic properties. The antiparasitic activity is thought to be due to the opening of a chlorine channel in the membrane of the neurons of the parasite under the effect of an increased release of the neuromediator GABA (gammaaminobutyric acid), inducing neuromuscular paralysis that may lead to the death of certain parasites. Ivermectin also interacts with other chlorine channels, especially those dependent on the neuromediator GABA (gammaaminobutyric acid).

Ivermectin is conventionally administered in the dermatological treatment of endoparasitic manifestations such as onchocerciasis and myiasis. U.S. Pat. No. 6,133,310 describes the use of ivermectin in the treatment of rosacea in order to reduce and eliminate the parasite Demodex folliculorum present on the skin of patients. U.S. Pat. No. 6,133,310 describes the use of ivermectin in the treatment of rosacea in order to reduce and eliminate the parasite Demodex folliculorum present on the skin of patients.

However, these treatments and compounds have drawbacks such as irritation and intolerance phenomena, especially when they are administered for a prolonged period. All current rosacea treatments seem only to be suppressive and not curative, acting especially on the pustulous spasms occurring during the inflammatory stage.

According to the National Rosacea Society an estimated 16 million Americans have rosacea, yet only a small fraction are being treated. Rosacea's etiology is currently under dispute in the dermatology community. Rosacea (roe-ZAY-she-uh) is a common skin condition that causes redness in your face and often produces small, red, pus-filled bumps. Left untreated, rosacea tends to worsen over time. Rosacea signs and symptoms may flare up for a period of weeks to months and then diminish before flaring up again. Rosacea can be mistaken for acne, an allergic reaction or other skin problems. While there is no cure for rosacea, current treatments can only help to control and reduce the signs and symptoms of the condition.

Rosacea is typically observed in individuals after the age of thirty as redness on the cheeks, nose, chin or forehead that may come and go. In some cases, rosacea may also occur on the neck, chest, scalp or ears. Over time the redness tends to become ruddier and more persistent, and visible blood vessels may appear. Left untreated, bumps and pimples often develop and in severe cases the nose may grow swollen and bumpy from excess tissue. This is the condition known as rhinophyma. In many rosacea patients the eyes are also affected, feeling irritated and appearing watery or bloodshot.

Although rosacea can affect all segments of the population, individuals with fair skin who tend to flush or blush easily are believed to be at greatest risk. The disease is more frequently diagnosed in women, but more severe symptoms tend to be seen in men. Rosacea can vary substantially from one individual to another and in most cases some rather than all of the potential signs and symptoms appear. According to a consensus committee and review panel of 17 medical experts worldwide, rosacea always includes at least one of the following primary signs, and various secondary signs and symptoms may also develop.

Primary Signs of rosacea include one or more of the following observable symptoms: (1) Flushing: Many people with rosacea have a history of frequent blushing or flushing. This facial redness may come and go, and is often the earliest sign of the disorder; (2) Persistent Redness Persistent facial redness is the most common individual sign of rosacea, and may resemble a blush or sunburn that does not go away; (3) Bumps and Pimples: Small red solid bumps or pus-filled pimples often develop. While these may resemble acne, blackheads are absent and burning or stinging may occur; (4) Visible Blood Vessels: In many people with rosacea, small blood vessels become visible on the skin.

Other Potential Signs and Symptoms include: (1) Eye Irritation. In many people with rosacea, the eyes may be irritated and appear watery or bloodshot, a condition known as ocular rosacea. The eyelids also may become red and swollen, and styes are common. Severe cases can result in corneal damage and vision loss without medical help. (2) Burning or Stinging. Burning or stinging sensations may often occur on the face. Itching or a feeling of tightness may also develop. (3) Dry Appearance. The central facial skin may be rough, and thus appear to be very dry. (4) Plaques. Raised red patches, known as plaques, may develop without changes in the surrounding skin. Skin (5) Thickening. The skin may thicken and enlarge from excess tissue, most commonly on the nose. This condition, known as rhinophyma, affects more men than women. (6) Swelling. Facial swelling, known as edema, may accompany other signs of rosacea or occur independently. (7) Signs Beyond the Face. Rosacea signs and symptoms may also develop beyond the face, most commonly on the neck, chest, scalp or ears.

Subtypes of rosacea: Subtype 1: (erythematotelangiectatic rosacea), characterized by flushing and persistent redness, and may also include visible blood vessels. Subtype 2: (papulopustular rosacea), characterized by persistent redness with transient bumps and pimples. Subtype 3: (phymatous rosacea), characterized by skin thickening, often resulting in an enlargement of the nose from excess tissue. Subtype 4: (ocular rosacea), characterized by ocular manifestations such as dry eye, tearing and burning, swollen eyelids, recurrent styes and potential vision loss from corneal damage.

Many patients experience characteristics of more than one subtype at the same time, and those often may develop in succession. While rosacea may or may not evolve from one subtype to another, each individual sign or symptom may progress from mild to moderate to severe. Early diagnosis and treatment are recommended.

As will be understood from the foregoing, methods of treating dermatological conditions, such as rosacea, are needed. Treatment methods are needed, for example, providing curative or long-term suppressive treatments capable of treating symptoms beyond the pustulous spasms which occur during the inflammation stage. Such treatment methods may be able to kill or inactivate the parasites or bacteria causing the allergic or vasomotor responses. Additionally, treatments exhibiting a combination enhanced efficacy and speed of treatment, low toxicity and reduced side effects would be useful and beneficial.

Chloroquine and/or hydroxychloroquine (Plaquenil) is human-approved with respect to a number of diseases and conditions, including for malaria.

### SUMMARY

Embodiments of the invention involve treatment of skin conditions by the topical and/or oral administration of chloroquine and/or hydroxychloroquine compounds. By effectively reducing or eliminating the population of Demodex mites in affected skin areas and areas where Demodex mites may exist, this treatment achieves a more complete remission of clinical signs and symptoms of the skin afflictions than conventional treatment approaches. In some embodiment, methods of the invention include treatment by administration of chloroquine and/or hydroxychloroquines so as to decrease the population of Demodex mites in afflicted regions of the skin and other regions of the skin not manifesting the conditions. Methods of the invention include treatment by repeated administration of chloroquine and/or hydroxychloroquines so as to maintain the population of Demodex mites in afflicted regions of the skin and other regions of the skin at levels low enough to prevent the skin affliction. Embodiments of the invention are useful for treating skin afflictions including common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis, rosacea and any combination of these.

Aspects of the invention include therapeutic and prophylactic methods for treating, managing and/or preventing dermatological conditions, by administration of a therapeutic agent having an chloroquine and/or hydroxychloroquine active ingredient. In some embodiments, for example, the method comprises topically administering chloroquine and/or hydroxychloroquine, to an individual afflicted with, or susceptible to, a dermatological condition such as rosacea, for example, by physically contacting regions of the skin and/or hair of the individual exhibiting symptoms of the dermatological condition with the chloroquine and/or hydroxychloroquine containing therapeutic agent and, optionally physically contacting regions of the skin and/or hair of the individual not manifesting symptoms with the chloroquine and/or hydroxychloroquine containing therapeutic agent. In an embodiment, for example, the chloroquine and/or hydroxychloroquine, such as diclorovos or a prodrug thereof, is topically administered to the individual by physically contacting at a dose and for an application period sufficient to significantly reduce the population of Demodex mites on and in treated region(s) of the individual, for example, by reducing the Demodex mite population on and in treated region(s) of the individual to a level equal to or less than 60%, optionally for some applications 90%, of the original population of Demodex mites. In an embodiment, for example, therapeutic agent is administered via a delivery means providing effective surface coverage and/or subdermal penetration of the chloroquine and/or hydroxychloroquine active agent for a treatment period sufficient to significantly reduce the population of Demodex mites on and in treated region(s) of the individual, for example, via topical administration via a body wash or shampoo, optionally applied to a significant portion (e.g., at least 50%) or substantially all (e.g., at least 90%) of the skin and hair of the individual.

The invention includes therapeutic and prophylactic methods including administering a therapeutic agent having an chloroquine and/or hydroxychloroquine active ingredient to an individual via a dosing regimen effective for controlling the population of Demodex mites, such as Demodex brevis mites and/or Demodex folliculorum mites, in treated regions of the skin and/or hair, for example, so as to be low enough to prevent, or ameliorate symptoms associated with, a dermatological condition, such as rosacea. Dosing regimens useful for the present methods include selection of one or more of the following: (1) the amount of chloroquine and/or hydroxychloroquine containing therapeutic agent administered to the individual, (2) the application period (e.g., contact time) in which the chloroquine and/or hydroxychloroquine containing therapeutic agent is provided in contact with the skin and/or hair of the individual, (3) the extent of the individual topically contacted with the chloroquine and/or hydroxychloroquine containing therapeutic agent (e.g., afflicted regions exhibiting symptoms of the condition and/or nonafflicted regions no exhibiting symptoms of the condition), (4) the frequency and duration of repeated administration of chloroquine and/or hydroxychloroquine containing therapeutic agent administered to the individual, and (5) administration of chloroquine and/or hydroxychloroquine containing therapeutic agent to the environment of the individual and any combination of these. The dosing regimens of the present invention are effective at reducing, and optionally maintaining, the population of Demodex mites on and in the individual at a level sufficient to prevent, manage or ameliorate the dermatological condition, for example, so as to reduce or eliminate symptoms of the dermatological condition.

In an aspect, the invention provides a method of treating a skin affliction comprising a step of orally administering or topically applying to an individual having the skin affliction an chloroquine and/or hydroxychloroquine in a dosage sufficient to inactivate Demodex brevis mites, Demodex folliculorum mites or both from hair follicles or skin of said individual, resulting in attenuation, amelioration and/or cessation of clinical symptoms associated with the skin affliction and caused, directly or indirectly, by the mites. In an aspect, a manifestation of the skin affliction may be an allergic and/or vasomotor responses to the mites that cause the skin affliction or symptoms thereof. In an embodiment, for example, afflicted regions and/or non-afflicted regions of the skin and/or hair follicles of the individual are physically contacted with the chloroquine and/or hydroxychloroquine, optionally in an amount to at least partially, or optionally entirely, fill pores in the skin and/or hair follicles of the treat.

In an embodiment, for example, said chloroquine and/or hydroxychloroquine is provided at said dosage sufficient to kill said Demodex brevis mites, Demodex folliculorum mites or both from hair follicles and/or skin of the individual contacted with said chloroquine and/or hydroxychloroquine. In an embodiment, for example, said chloroquine and/or hydroxychloroquine is provided to said individual at said dosage is sufficient to provide a reduction in the population of said Demodex brevis mites, Demodex folliculorum mites or both from said hair follicles or skin contacted with said chloroquine and/or hydroxychloroquine greater than or equal to 80%. In an embodiment, for example, said dosage of the chloroquine and/or hydroxychloroquine is sufficient to provide for said reduction in the population of said Demodex brevis mites, Demodex folliculorum mites or both over a time interval less than or equal to 1 month. In an embodiment, for example, the method further comprises re-applying or re-administering said chloroquine and/or hydroxychloroquine in a dosage sufficient to maintain said reduction in said population of said Demodex brevis mites, Demodex folliculorum mites or both over a time interval of between about 10 to 120 days, or greater than or equal to two months. In an aspect, the re-applying or re-administering is timed to a life cycle property of the mites being controlled. For example, the time period may be greater than a time for an egg to hatch but less than the time for an adult mite to provide a fertilized egg. In an aspect, the time between consecutive administrations may be between 3 and 10 days or may be between 3 and 7 days. In an aspect, the time between consecutive administrations is selected so that administration is about bi-weekly or is bi-weekly. In an embodiment, for example, said chloroquine and/or hydroxychloroquine is provide at said dosage sufficient to render said Demodex brevis mites, Demodex folliculorum mites or both incapable of reproducing on or in hair follicles and/or skin of the individual contacted with said chloroquine and/or hydroxychloroquine. In an embodiment, for example, said chloroquine and/or hydroxychloroquine is provided at said dosage sufficient to at least partially, and optionally completely, fill pores of said skin, hair follicles or both of the individual contacted with said chloroquine and/or hydroxychloroquine. In an aspect, the incapable of reproducing may reflect killing of adult mites. In an aspect, the incapable of reproducing may reflect killing of pre-adult mites, such as larvae. In an aspect, the incapable of reproducing may reflect making the eggs non-viable, so that larvae does not emerge from eggs or any emergent from the eggs cannot grow into a mite capable of reproducing.

The present methods are versatile and useful for treatment of a range of dermatological conditions, for example, skin conditions affecting the facial skin, eyelids or both. In an embodiment, for example, the skin affliction is one or more of common acne, seborrheic dermatitis, perioral dermatitis, an acneform rash, transient acantholytic dermatosis, acne necrotica milliaris, psoriasis, steroid induced dermatitis, primary irritation dermatitis or rosacea. In an embodiment, for example, the skin condition is rosacea. In an embodiment, for example, the skin affliction is erythematotelangiectatic rosacea, papulopustular rosacea, phymatous rosacea, ocular rosacea or rhinophyma.

A range of active ingredients are useful in the present methods, particularly those active agents exhibiting a combination of useful skin penetration, efficacy for inactivating Demodex mites, and low toxicity. In an embodiment, for example, said chloroquine and/or hydroxychloroquine is a miticide or insecticide. In an embodiment, for example, said chloroquine and/or hydroxychloroquine kills Demodex brevis mites, Demodex folliculorum mites or both. In an embodiment, for example, said chloroquine and/or hydroxychloroquine kills larva or eggs of said Demodex brevis mites, Demodex folliculorum mites or both.

Certain methods of the invention further comprise administration of the chloroquine and/or hydroxychloroquine to the individual in a manner providing delivery to tissue within and/or beneath the outer layer of the skin, such as delivery into the pores of the skin and/or to regions beneath the epidermis. In an embodiment, for example, said chloroquine and/or hydroxychloroquine is transported into an epidermis or a subdermal region upon contact with said hair follicles and/or skin of the individual, optionally exhibiting a transport rate into said epidermis or a subdermal region upon contact with said hair follicles and/or skin of the individual to provide chloroquine and/or hydroxychloroquine contact with the mite in a time interval that is less than or equal to 1 minute. In an embodiment, for example, the chloroquine and/or hydroxychloroquine is characterized by a biological half-life less than or equal to 30 minutes. Certain formulations of the topical chloroquine and/or hydroxychloroquines useful with the methods of the invention optionally comprise one or more compositions that increase a permeability of the skin, such as dimethyl sulfoxide (DMSO).

Chloroquine and/or hydroxychloroquine active agents of the invention may be provided in a variety of forms useful for formulation, administration and delivery. In an embodiment, the chloroquine and/or hydroxychloroquine active ingredient is administered topically to the individual in need. In an embodiment, for example, the chloroquine and/or hydroxychloroquine topically applied as formulated in a carrier lotion, cream, soap, wash, shampoo or gel. In an embodiment, for example, the chloroquine and/or hydroxychloroquine has a concentration in the topically applied lotion, cream, soap, wash, shampoo or gel selected from the range 0.001 to 5 percent by weight, optionally for some applications selected from the range 0.001 to 5 percent by weight, 0.01 to 1 percent by weight, 0.5% to 2% by weight, or about 1% by weight. In an embodiment, for example, the chloroquine and/or hydroxychloroquine has a concentration in the topically applied lotion, cream, soap, wash, shampoo or gel selected from the range 2 to 5 percent by weight, optionally for some applications selected from the range 5 to 10 percent by weight, 10 to 15 percent by weight, 15% to 20% by weight, about 2% by weight, about 5% by weight, about 10% by weight, between 0.001% to 15%, up to 15% by weight, about 15% by weight, up to 20% by weight, or about 20% by weight. In an embodiment, for example, the chloroquine and/or hydroxychloroquine in the topically applied lotion, cream, soap, wash, shampoo or gel is provided in a lowest concentration effective for killing the Demodex brevis mites, Demodex folliculorum mites or both. In an embodiment, for example, a method of the invention further comprises providing a dosage of said chloroquine and/or hydroxychloroquine in the topically applied lotion, cream, soap, wash, shampoo or gel less than 150 mg/kg of body mass. In an embodiment, for example, a method of the invention further comprises providing a dosage of said chloroquine and/or hydroxychloroquine in the topically applied lotion, cream, soap, wash, shampoo or gel selected from the range of 0.01 mg per kg of body mass to 50 mg/kg of body mass, or about 0.1 mg/kg of body mass to 1.5 mg/kg of body mass, or about 0.6 mg/kg of body mass, including for an chloroquine and/or hydroxychloroquine.

In an embodiment, the therapeutic agent containing the chloroquine and/or hydroxychloroquine active ingredient is topically administered to the individual during an administration period sufficient to provide high coverage (e.g., 80% or greater) of the treated regions of the skin and hair and/or subdermal penetration of the chloroquine and/or hydroxychloroquine active ingredient to a least a portion of and optionally substantially all (e.g., 80% or greater) of the treated regions of the skin. In an embodiment, the therapeutic agent containing the chloroquine and/or hydroxychloroquine active ingredient is topically administered to the individual for an administration period selected from the range of 1 second to 10 minutes, and optionally for some embodiments 5 seconds to 5 minutes and optionally for some embodiments 10 seconds to 1 minute. In an embodiment, said dosage of chloroquine and/or hydroxychloroquine in the topically applied lotion, cream, soap, wash, shampoo or gel is a lowest dose effective for killing the Demodex mites, for example, to an extent useful for preventing or ameliorating the affliction. In an embodiment, for example, the topically applied chloroquine and/or hydroxychloroquine is encapsulated, for example, in a vesicles, microliposomes or micelles. In an embodiment, for example, the topically applied chloroquine and/or hydroxychloroquine is provided as an emulsion, optionally a nanoemulsion, in said topically applied lotion, cream, soap, wash, shampoo or gel.

Methods of the invention include repeated treatment of the skin and/or hair follicles of the individual, for example, to systematically reduce and/or maintain the population of the Demodex mites at a level resulting in prevention, elimination or amelioration of the affliction or symptoms thereof. In an embodiment, for example, a method of the invention further comprises topically applying said chloroquine and/or hydroxychloroquine to skin areas affected by the skin affliction. In an embodiment, for example, a method of the invention further comprises topically applying said chloroquine and/or hydroxychloroquine to skin areas not affected by the skin affliction. In an embodiment, for example, a method of the invention further comprises topically applying said chloroquine and/or hydroxychloroquine to skin and hair areas of the body where Demodex brevis mite or Demodex folliculorum mites are present. In an embodiment, for example, a method of the invention further comprises topically applying said chloroquine and/or hydroxychloroquine to all skin areas of said individual. In an embodiment, for example, a method of the invention further comprises topically applying said chloroquine and/or hydroxychloroquine to all hair areas of said individual.

Treatment methods of the invention may also include additional steps to avoid or reduce the extent of repopulation of Demodex mites on the individual undergoing treatment. In an embodiment, for example, a method of the invention further comprises a step of applying the chloroquine and/or hydroxychloroquine to the individual's clothing, linens or both clothing and linens. In an embodiment, a method of the invention further comprises a step of orally administering or topically applying the chloroquine and/or hydroxychloroquine to others having contact with the individual in a dosage sufficient to inactivate Demodex brevis mites, Demodex folliculorum mites or both from hair follicles or skin of the others, for example, wherein such others include household members, children, spouses, partners, family members or pets.

In an embodiment, for example, the topically applied chloroquine and/or hydroxychloroquine is applied to the hair follicles and skin of the individual. In an aspect, for example, the topically applied chloroquine and/or hydroxychloroquine penetrates an outer layer of the skin of the individual, thereby exposing the Demodex brevis mites, Demodex folliculorum mites or both present below the outer layer of the skin to the chloroquine and/or hydroxychloroquine, for example, to a depth below the outer layer of the skin selected over the range of 1 μm to 3 mm. In an embodiment, for example, the topically applied chloroquine and/or hydroxychloroquine penetrates to a subdermal region of the skin of the individual, such as the dermis or subcutis regions, thereby exposing the Demodex brevis mites, Demodex folliculorum mites or both present in the subdermal region of the skin to the chloroquine and/or hydroxychloroquine.

Methods of the invention include dosing and re-administration regimens effective for reducing and maintaining the population of Demodex mites to provide prevention, elimination or amelioration of the skin conditions or symptoms thereof. In an embodiment, for example, the chloroquine and/or hydroxychloroquine is orally administered or topically applied in a continued intermittent regime sufficient for prophylactic control of Demodex mite population in the hair follicles and/or skin of the individual.

In an embodiment, for example, the topically applied chloroquine and/or hydroxychloroquine is applied to affected skin areas at least once and not more than twice daily for a period of two to six weeks. In an embodiment, for example, the topically applied chloroquine and/or hydroxychloroquine is applied to the affected skin areas and/or to non-affected skin areas during a first application period, thereby inactivating said Demodex brevis mites, Demodex folliculorum mites or both from the hair follicles in the skin of the individual. In an embodiment, for example, the topically applied chloroquine and/or hydroxychloroquine is further applied to the affected skin areas and/or to non-affected skin areas during a second application period, thereby inactivating said Demodex brevis mites, Demodex folliculorum mites or both from the hair follicles and/or skin of the individual that have matured from a larval form and/or an egg form present on and/or in the skin during or after the first application period. In an embodiment, for example, the topically applied chloroquine and/or hydroxychloroquine is further applied to the affected skin areas and/or to non-affected skin areas during a third application period, thereby inactivating said Demodex brevis mites, Demodex folliculorum mites or both from the hair follicles and or skin of the individual Demodex brevis and/or Demodex folliculorum mites that have matured from a larval form and/or an egg form present on and/or in the skin and/or the hair follicles during or after the first application period and/or the second application period.

In an embodiment, for example, the first application period and the second application period are separated by at least five days and not more than ten days, and optionally for some embodiments, the first application period and the second application period are separated by at least seven days. In an embodiment, for example, the first application period and the second application period are separated by a time sufficient to allow larva of said Demodex brevis mites, Demodex folliculorum mites or both to mature into an adult form and/or to allow eggs of said Demodex brevis mites, Demodex folliculorum mites or both to mature into the adult form. In an embodiment, for example, the second application period and the third application period are separated by at least five days and not more than ten days, and optionally for some embodiments, the second application period and the third application period are separated by at least seven days. In an embodiment, for example, the second application period and the third application period are separated by a time sufficient to allow time sufficient to allow larva of said Demodex brevis mites, Demodex folliculorum mites or both to mature into an adult form and/or to allow eggs of said Demodex brevis mites, Demodex folliculorum mites or both to mature into the adult form.

In an aspect, the chloroquine and/or hydroxychloroquine active ingredient is administered orally to the individual in need. In an embodiment, for example, the orally administered chloroquine and/or hydroxychloroquine is administered as an oral dose equal to or less than 150 mg per kg of body mass. In an embodiment, for example, the orally administered chloroquine and/or hydroxychloroquine is administered as an oral dose selected from the range of 0.01 mg per kg of body mass and 50 mg per kg of body mass. In an embodiment, for example, the orally administered chloroquine and/or hydroxychloroquine is administered as an oral dose of a lowest dose effective for killing the Demodex mites. In an embodiment, for example, the orally administered chloroquine and/or hydroxychloroquine is administered as a daily dose of 1 to 20 mg per kg of body mass, optionally 10 mg per kg of body mass. In an embodiment, for example, the orally administered chloroquine and/or hydroxychloroquine is administered as a daily dose 1 to 10 mg per kg of body mass, optionally of 7.5 mg per kg of body mass. In an embodiment, for example, the orally administered chloroquine and/or hydroxychloroquine is administered as a three times per day dose of 1 to 10 mg per kg of body mass, optionally 5 mg per kg of body mass. In an embodiment, for example, the orally administered chloroquine and/or hydroxychloroquine is repeated two to four times with spacing of three to seven days between them. In an embodiment, for example, the orally administered chloroquine and/or hydroxychloroquine is formulated as a prodrug or pharmaceutically acceptable salt or ester.

In an aspect, methods of the invention are useful for eliminating the presence and/or reducing population of bacteria originating from Demodex mites on the skin and/or hair follicles of the individual, such as eliminating or reducing bacteria that result in allergic and/or vasomotor responses that cause the skin affliction in the individual and symptoms thereof. In an embodiment, for example, the inactivation of the Demodex brevis and/or Demodex folliculorum mites from hair follicles and/or skin of the individual results in a reduction in population of one or more bacteria in the hair follicles and/or skin of the individual. In an embodiment, for example, the allergic and/or vasomotor responses to the mites result from a presence of one or more bacteria associated with the mites in the hair follicles and/or skin of the individual. In an embodiment, for example, the one or more bacteria comprise one or more bacteria from the genus staphylococcus or from the genus bacillus. In an embodiment, for example, the one or more bacteria comprise bacillus oleronius bacteria. In an embodiment, for example, the one or more bacteria comprise Staphylococcus epidermidis bacteria. In an embodiment, for example, the one or more bacteria are present in a digestive system of the Demodex brevis and/or Demodex folliculorum mites.

In an aspect, the invention provides a method of treating a skin affliction comprising a step of topically applying an active ingredient in a dosage to an individual having the skin affliction to inactivate Demodex brevis mites, Demodex folliculorum mites or both from hair follicles or skin of said individual, resulting in cessation of the manifestations of allergic and/or vasomotor responses to the mites that cause symptoms and signs of the skin affliction in the individual resulting in amelioration or cessation of the manifestations of allergic and/or vasomotor responses to the mites that cause the skin affliction or symptoms thereof, wherein the topically applied active ingredient is applied to skin areas affected by the skin affliction and to skin areas not affected by the skin affliction. In an embodiment, for example, afflicted regions and/or non-afflicted regions of the skin and/or hair follicles of the individual are physically contacted with the active ingredient, optionally in an amount to at least partially fill pores in the skin and/or hair follicles.

In this manner, the treatment may correspond more to a whole-body treatment wherein the active ingredient is formulated in a shampoo or body-wash that can be applied to a large portion of the individual's skin in a rapid and effective manner. In an embodiment, the shampoo or body-wash is applied to at least 50% of the total surface area of skin of the individual, and may, therefore, include both skin areas exhibiting a symptom and other skin areas that are do not indicate a symptom. In an aspect, the total surface area is selected from a range that is greater than 50% and up to 100% of the total skin surface area.

In an embodiment, for example, the topically applied active ingredient is applied to all skin of the individual to inactivate Demodex brevis mites, Demodex folliculorum mites or both from all skin of the individual. In an embodiment, for example, the active ingredient comprises a chloroquine and/or hydroxychloroquine. In an embodiment, for example, the active ingredient comprises a miticide or insecticide. In an embodiment, for example, the chloroquine and/or hydroxychloroquine kills Demodex brevis mites, Demodex folliculorum mites or both. In an embodiment, for example, the chloroquine and/or hydroxychloroquine kills larva or eggs of said Demodex brevis mites, Demodex folliculorum mites or both. In an embodiment, for example, the skin affliction comprises one or more of common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis or rosacea.

### Statements Regarding Chemical Compounds and Nomenclature

In an embodiment, a composition or compound used with the methods of the invention is isolated or purified. In an embodiment, an isolated or purified compound is at least partially isolated or purified as would be understood in the art. In an embodiment, the composition or compound of the invention has a chemical purity of 95%, optionally for some applications 99%, optionally for some applications 99.9%, optionally for some applications 99.99%, and optionally for some applications 99.999% pure.

Many of the compounds used in the methods of the invention contain one or more ionizable groups. Ionizable groups include groups from which a proton can be removed (e.g., —COOH) or added (e.g., amines) and groups which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

Hydroxychloroquine was approved for medical use in the United States in the 1950s and is on the World Health Organization's List of Essential Medicines, with the chemical structure:

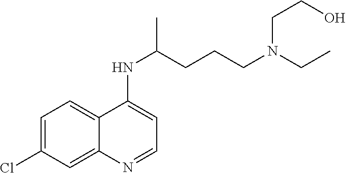 [SMILES: CCN(CCO)CCCC(C)Nc1ccnc2cc(Cl)ccc12]

Chloroquine has the structural formula:

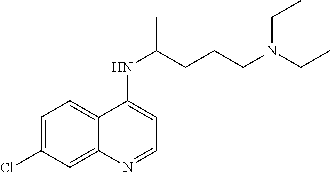 [SMILES: CCN(CC)CCCC(C)Nc1ccnc2cc(Cl)ccc12]

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

As used herein, the term “halo” refers to a halogen group such as a fluoro (—F), chloro (—Cl), bromo (—Br), iodo (—I) or astato (—At).

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenylgroups can also carry alkyl groups. Cycloalkenylgroups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —ON;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

The compounds used in the methods of this invention can contain one or more chiral centers. Accordingly, this invention is intended to include racemic mixtures, diasteromers, enantiomers, tautomers and mixtures enriched in one or more stereoisomer. The scope of the invention as described and claimed encompasses the racemic forms of the compounds as well as the individual enantiomers and non-racemic mixtures thereof.

“Inactivate” in the context of the methods provided herein refers to a process by which an organism or microorganism is rendered incapable of reproducing, growing and/or surviving. In some embodiments, active agents of the present invention, such as chloroquine and/or hydroxychloroquine, inactivate Demodex brevis mites, Demodex folliculorum mites and/or larva and/or eggs thereof. In some embodiments, inactivation results in death or elimination of Demodex brevis mites, Demodex folliculorum mites and/or larva and/or eggs thereof. In other embodiments, the inactivation selectively targets one phase of the mite lifecycle. In this manner, multiple applications that are spaced in time can be beneficial in controlling mite population, so that mites at different stages of the life cycle may then be appropriately targeted. For example, an application that targets an adult mite may not effectively control larvae or eggs can be addressed by a subsequent therapeutic application of the chloroquine and/or hydroxychloroquine timed to ensure that a subsequent generation of mites from an earlier life cycle phase are targeted. Similarly, applications that target eggs or larvae may be timed to target subsequent generations of eggs or larvae. Accordingly, inactivate includes aspects where there is only partial inactivation of a mite population, but that a more complete inactivation occurs with subsequently timed treatments.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, -32-cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other pharmaceutically acceptable salts may be found, for example, in Stahl et al., Handbook of Pharmaceutical Salts: Properties, Selection, and Use, Wiley-VCH; Verlag Helvetica Chimica Acta, Zurich, 2002. (ISBN 3-906390-26-8). Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

Exemplary claims of the invention include any one or more of:

1. A method of treating a skin affliction, the method comprising the step of administering to the individual chloroquine and/or hydroxychloroquine in a dosage sufficient to inactivate at least a portion of Demodex mites in the human body, wherein the inactivation results in attenuation or cessation of one or more symptoms associated with the skin affliction.
2. The method of claim 1, wherein substantially all Demodex mites in the human body are inactivated.
3. The method of claim 1 or 2, wherein the one or more clinical symptom is associated with an allergic and/or vasomotor response to the Demodex mites that cause the skin affliction or symptoms thereof.
4. The method of any of claims 1-3, wherein the chloroquine and/or hydroxychloroquine is provided at said dosage sufficient to kill said Demodex mites from hair follicles and/or skin of the individual contacted with said chloroquine and/or hydroxychloroquine.
5. The method of any of claims 1-5, wherein said dosage is sufficient to provide a reduction in the population of said Demodex mites from said hair follicles or skin contacted with said chloroquine and/or hydroxychloroquine that is greater than or equal to 80%.
6. The method of claim 5, wherein said reduction in the population is by one or more of: killing adult mites; killing mite larva; or affecting a fertilized egg to prevent emergence of a viable larva.
7. The method of claim 5, wherein said dosage is sufficient to provide for said reduction in the population of said Demodex mites over a time interval that is up to 1 month.
8. The method of claim 5, further comprising re-applying or re-administering said chloroquine and/or hydroxychloroquine in a dosage sufficient to maintain said reduction in said population of said Demodex mites over a time interval greater than or equal to two months.
9. The method of claim 8, wherein the re-applying or re-administering is synchronized with a life cycle of the mites so as to inactivate a second generation of mites that were in an egg or larval form during the initial orally administering or topically applying step and before a substantial portion of the second generation of mites are capable of reproducing.
10. The method of claim 9, further comprising re-applying or re-administering a second time, wherein the time interval between temporally adjacent applications is independently selected over a time that is greater than or equal to 3 days and less than or equal to 7 days.
11. The method of claim 1, wherein said dosage is sufficient to render said Demodex mites incapable of reproducing in or on hair follicles and/or skin of the individual contacted with said chloroquine and/or hydroxychloroquine.
12. The method of claim 1, wherein said chloroquine and/or hydroxychloroquine is provided at said dosage sufficient to fill pores of said skin, hair follicles or both of the individual contacted with said chloroquine and/or hydroxychloroquine.
13. The method of any of claims 1-12, wherein the skin affliction affects facial skin or eyelids, or both.
14. The method of any of claims 1-12, wherein the skin affliction is one or more of common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis or rosacea.
15. The method of any of claims 1-14, wherein the skin affliction is rosacea.
16. The method of any of claims 1-14, wherein the skin affliction is erythematotelangiectatic rosacea, papulopustular rosacea, phymatous rosacea, ocular rosacea or rhinophyma.
17. The method of any of claims 1-14, wherein the skin affliction is acne vulgaris.
18. The method of any of claims 1-17, wherein said chloroquine and/or hydroxychloroquine is a miticide or an insecticide.
19. The method of claim 18, wherein said chloroquine and/or hydroxychloroquine kills Demodex brevis mites, Demodex folliculorum mites or both.
20. The method of claim 18, wherein said chloroquine and/or hydroxychloroquine kills larva or eggs of said Demodex brevis mites, Demodex folliculorum mites or both.
21. The method of claim 1, wherein said chloroquine and/or hydroxychloroquine is transported into an epidermis or a subdermal region upon contact with said hair follicles and/or skin of the individual.
22. The method of claim 21, wherein said chloroquine and/or hydroxychloroquine is characterized by a transport rate into said epidermis or a subdermal region upon contact with said hair follicles and/or skin of the individual equal to or greater than 0.1 microns per second.
23. The method of any of claims 1-22, wherein the chloroquine and/or hydroxychloroquine is characterized by a biological half-life less than or equal to 15 minutes.
24. The method of claim 1, wherein the chloroquine and/or hydroxychloroquine topically applied as formulated in a carrier lotion, cream, soap, wash, shampoo or gel.
25. The method of claim 24, wherein the chloroquine and/or hydroxychloroquine has a concentration in the topically applied carrier lotion, cream, soap, wash, shampoo or gel selected from the range 0.001 to 5 percent by weight.
26. The method of claim 24, wherein the chloroquine and/or hydroxychloroquine in the topically applied carrier lotion, cream, soap, wash, shampoo or gel is provided in a lowest concentration effective for killing the Demodex brevis mites, Demodex folliculorum mites or both.
27. The method of claim 24, comprising providing a dosage of said chloroquine and/or hydroxychloroquine in the topically applied carrier lotion, cream, soap, wash, shampoo or gel less than 150 mg/kg of body mass.
28. The method of claim 24, comprising providing a dosage of said chloroquine and/or hydroxychloroquine in the topically applied carrier lotion, cream, soap, wash, shampoo or gel selected from the range of 0.01 mg per kg of body mass to 50 mg/kg of body mass.
29. The method claim 24, wherein said dosage of chloroquine and/or hydroxychloroquine in the topically applied carrier lotion, cream, soap, wash, shampoo or gel is a lowest dose effective for killing the Demodex mites.
30. The method of claim 24, wherein the topically applied chloroquine and/or hydroxychloroquine is encapsulated inside microliposomes or micelles.
31. The method of claim 24, wherein the topically applied chloroquine and/or hydroxychloroquine is provided as an emulsion in said topically applied lotion, cream, soap, wash, shampoo or gel.
32. The method of claim 1, comprising topically applying said chloroquine and/or hydroxychloroquine to skin areas exhibiting the clinical symptom.
33. The method of claim 32, further comprising topically applying said chloroquine and/or hydroxychloroquine to skin areas not exhibiting the clinical symptom.
34. The method of any of claims 1-33, further comprising topically applying said chloroquine and/or hydroxychloroquine to skin and hair areas of the body where Demodex brevis mite or Demodex folliculorum mites are present.
35. The method of any of claims 1-34, further comprising topically applying said chloroquine and/or hydroxychloroquine to all skin areas of said individual.
36. The method of any of claims 1-35, further comprising topically applying said chloroquine and/or hydroxychloroquine to all hair areas of said individual.
37. The method of any of claims 1-36, further comprising a step of applying the chloroquine and/or hydroxychloroquine to the individual's clothing, linens or both clothing and linens.
38. The method of any of claims 1-36, further comprising a step of orally administering or topically applying the chloroquine and/or hydroxychloroquine to others having contact with the individual in a dosage sufficient to inactivate Demodex brevis mites, Demodex folliculorum mites or both from hair follicles or skin of the others.
39. The method of claim 38, wherein the others comprise household members, children, spouses, partners, family members or pets.
40. The method of any of claims 1-39, wherein the topically applied chloroquine and/or hydroxychloroquine is applied to the hair follicles and skin of the individual.
41. The method of any of claims 1-40, wherein the topically applied chloroquine and/or hydroxychloroquine penetrates an outer layer of the skin of the individual, thereby exposing the Demodex brevis mites, Demodex folliculorum mites or both present below the outer layer of the skin to the chloroquine and/or hydroxychloroquine.
42. The method of any of claims 1-41, wherein the topically applied chloroquine and/or hydroxychloroquine penetrates to a subdermal region of the skin of the individual, thereby exposing the Demodex brevis mites, Demodex folliculorum mites or both present in the subdermal region of the skin to the chloroquine and/or hydroxychloroquine.
43. The method of any of claims 1-42, wherein the topically applied chloroquine and/or hydroxychloroquine is applied to affected skin areas at least once and not more than twice daily for a period of two to twelve weeks.
44. The method of any of claims 1-43, wherein the topically applied chloroquine and/or hydroxychloroquine is applied to the affected skin areas and/or to non-affected skin areas during a first application period, thereby inactivating said Demodex brevis mites, Demodex folliculorum mites or both from the hair follicles in the skin of the individual.
45. The method of claim 44, wherein the topically applied chloroquine and/or hydroxychloroquine is further applied to the affected skin areas and/or to non-affected skin areas during a second application period, thereby inactivating said Demodex brevis mites, Demodex folliculorum mites or both from the hair follicles and/or skin of the individual that have matured from a larval form and/or an egg form present on and/or in the skin during or after the first application period.
46. The method of claim 45, wherein the topically applied chloroquine and/or hydroxychloroquine is further applied to the affected skin areas and/or to non-affected skin areas during a third application period, thereby inactivating said Demodex brevis mites, Demodex folliculorum mites or both from the hair follicles and or skin of the individual Demodex brevis and/or Demodex folliculorum mites that have matured from a larval form and/or an egg form present on and/or in the skin and/or the hair follicles during or after the first application period and/or the second application period.
47. The method of claim 45, wherein the first application period and the second application period are separated by at least three days and not more than seven days.
48. The method of claim 45, wherein the first application period and the second application period are separated by at least three days.
49. The method of claim 45, wherein the first application period and the second application period are separated by a time sufficient to allow larva of said Demodex brevis mites, Demodex folliculorum mites or both to mature into an adult form and/or to allow eggs of said Demodex brevis mites, Demodex folliculorum mites or both to mature into the adult form.
50. The method of claim 46, wherein the second application period and the third application period are separated by at least three days and not more than seven days.
51. The method of claim 46, wherein the second application period and the third application period are separated by at least three days.
52. The method claim 46, wherein the second application period and the third application period are separated by a time sufficient to allow time sufficient to allow larva of said Demodex brevis mites, Demodex folliculorum mites or both to mature into an adult form and/or to allow eggs of said Demodex brevis mites, Demodex folliculorum mites or both to mature into the adult form.
53. The method of any of claims 1-52, wherein the chloroquine and/or hydroxychloroquine is orally administered or topically applied in a continued intermittent regime sufficient for prophylactic control of Demodex mite population in the hair follicles and/or skin of the individual.
54. The method of any of claims 1-11, wherein the administering is by oral administration at an oral dose equal to or less than 150 mg per kg of body mass.
55. The method of claim 54, wherein the oral dose is selected from the range of 0.01 mg per kg of body mass and 50 mg per kg of body mass.
56. The method of any of claims 1-11, wherein the chloroquine and/or hydroxychloroquine is orally administered at an oral dose that is a lowest dose effective for killing the Demodex mites.
57. The method of claim 54, wherein the orally administered chloroquine and/or hydroxychloroquine is administered as a daily dose of 1 to 20 mg per kg of body mass, optionally 10 mg per kg of body mass.
58. The method of claim 54, wherein the orally administered chloroquine and/or hydroxychloroquine is administered as a daily dose of 1 to 10 mg per kg of body mass, optionally 7.5 mg per kg of body mass.
59. The method of claim 54, wherein the orally administered chloroquine and/or hydroxychloroquine is administered as a three times per day dose of 1 to 10 mg per kg of body mass, optionally 5 mg per kg of body mass.
60. The method of any of claims 1-11, wherein the orally administered chloroquine and/or hydroxychloroquine is repeated two to four times with spacing of three to seven days between them.
61. The method of claim 54, wherein the orally administered chloroquine and/or hydroxychloroquine is formulated as a prodrug or pharmaceutically acceptable salt or ester.
62. The method of any of claims 1-11, wherein the inactivation of the Demodex brevis and/or Demodex folliculorum mites from hair follicles and/or skin of the individual results in a reduction in population of one or more bacteria in the hair follicles and/or skin of the individual.
63. The method of claim 2, wherein the manifestations of allergic and/or vasomotor responses to the mites result from a presence of one or more bacteria associated with the mites in the hair follicles and/or skin of the individual.
64. The method of claim 62, wherein the one or more bacteria comprise one or more bacteria from the genus staphylococcus or from the genus bacillus.
65. The method of claim 62, wherein the one or more bacteria comprise bacillus oleronius.
66. The method of claim 62, wherein the one or more bacteria comprise Staphylococcus epidermidis.
67. The method of any of claim 62, wherein the one or more bacteria are present in a digestive system of the Demodex brevis and/or Demodex folliculorum mites.
68. A method of treating a skin affliction comprising a step of topically applying an active ingredient in a dosage to an individual having the skin affliction to inactivate Demodex brevis mites, Demodex folliculorum mites or both from hair follicles or skin of said individual, resulting in attenuation or cessation of a clinical symptom associated with the skin affliction, wherein the topically applied active ingredient is applied to skin areas exhibiting the clinical symptom and to skin areas not exhibiting the clinical symptom.
69. The method of claim 68, wherein the clinical symptom is a manifestation of allergic and/or vasomotor responses to the Demodex brevis mites, Demodex folliculorum mites or both Demodex brevis mites and Demodex folliculorum mites.
70. The method of claim 68, wherein the topically applied active ingredient is applied to all skin of the individual to inactivate Demodex brevis mites, Demodex folliculorum mites or both from all skin of the individual.
71. The method of any of claims 68-70, wherein the active ingredient is formulated in a shampoo or body-wash and the shampoo or body-wash is applied to at least 50% of the total surface area of skin of the individual.
72. The method of any of claims 68-71, wherein the active ingredient comprises hydroxychloroquine.
73. The method of any of claims 68-72, wherein said chloroquine and/or hydroxychloroquine kills Demodex brevis mites, Demodex folliculorum mites or both.
74. The method of any of claims 68-72, wherein said chloroquine and/or hydroxychloroquine kills larva or eggs of said Demodex brevis mites, Demodex folliculorum mites or both.
75. The method of any of claim 68-74, wherein the skin affliction comprises one or more of common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis or rosacea.
76. The method of claim 1 or 68, further comprising co-administration or post-administration with intense light or intense pulsed light.
77. The method of claim 1 or 68, wherein the method of treating is for a human patient.
78. A method for determining presence or absence of a skin affliction caused by Demodex brevis mites, Demodex folliculorum mites, or both in a patient, the method comprising the steps of:
applying to a skin application region of the patient a chloroquine and/or hydroxychloroquine having an inactivation activity for Demodex brevis mites, Demodex folliculorum mites, or both for an application time; and
observing the skin application region for a flare-up response;
wherein an observable flare-up response indicates presence of the skin affliction caused by Demodex brevis mites, Demodex folliculorum mites, or both; and
a no observable flare-up response indicates absence of the skin affliction caused by Demodex brevis mites, Demodex folliculorum mites, or both.
79. The method of claim 78, further comprising the step of, for a patient having the observable flare-up response, treating the patient by:
topically applying to the patient the chloroquine and/or hydroxychloroquine in a dosage sufficient to inactivate mites from hair follicles or skin of said individual, resulting in attenuation or cessation of a one or more clinical symptom of the skin affliction.
80. The method of claim 79, wherein the application time is selected from a range that is greater than or equal to 10 seconds and less than or equal to 2 minutes.
81. The method of any of claims 79-80, wherein the application region has a surface area that is less than or equal to 10 cm².
82. The method of any of claims 79-81, wherein the applying step comprises applying a substrate layer having a contact face that supports the chloroquine and/or hydroxychloroquine to the skin application region.
83. The method of any of claims 79-82, wherein the flare-up response comprises an immune response and/or a vasomotor response.
84. The method of claim 82, wherein the flare-up response is one or more of localized reddening, discoloration, swelling, itching, or irritation.
85. A method of alleviating in a subject symptoms associated with an autoimmune disease caused by Demodex organisms, the method comprising administering chloroquine and/or hydroxychloroquine to the subject having the autoimmune disease in a dosage sufficient to inactivate at least a portion of the Demodex organisms.
86. The method of claim 85, further comprising the step of identifying a subject in need of alleviating symptoms associated with the autoimmune disease caused by demodex organisms.
87. The method of claim 85 or 86, further comprising the step of monitoring whether the subject experiences symptom alleviations.
88. The method of any of claims 85-84, wherein the administration is topically, orally, or intravenously providing the chloroquine and/or hydroxychloroquine to the subject.
89. The method of any of claims 85-88, wherein the administration is a topical administration.
90. The method of claim 89, wherein the topical administration comprises applying the chloroquine and/or hydroxychloroquine to the face and hair follicles of the face and head.
91. The method of claim 89, wherein the topical administration comprises applying the chloroquine and/or hydroxychloroquine to substantially the entire body.
92. The method of any of claims 85-91, further comprising the step of sampling demodex levels in the patient after the administering step.
93. The method of claim 92, wherein the sampling step comprises one or more of visualization of a skin surface, swabbing a skin surface, removing hair, a skin surface biopsy using an adhesive, a skin biopsy, or staining to visualize demodex such as by Löffler's alkaline methylene blue staining.
94. The method of claim 24, wherein the chloroquine and/or hydroxychloroquine has a concentration in the topically applied carrier lotion, cream, soap, wash, shampoo or gel of between 0.001 percent to 15 percent by weight.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is an illustration of Demodex folliculorum and Demodex brevis mites, including relative sizes and locations in the skin, hair follicles and glands. FIG. 1 is adapted from Murube J. (2015) Demodex hominis. Ocul Surf. 13(3): 181-186.

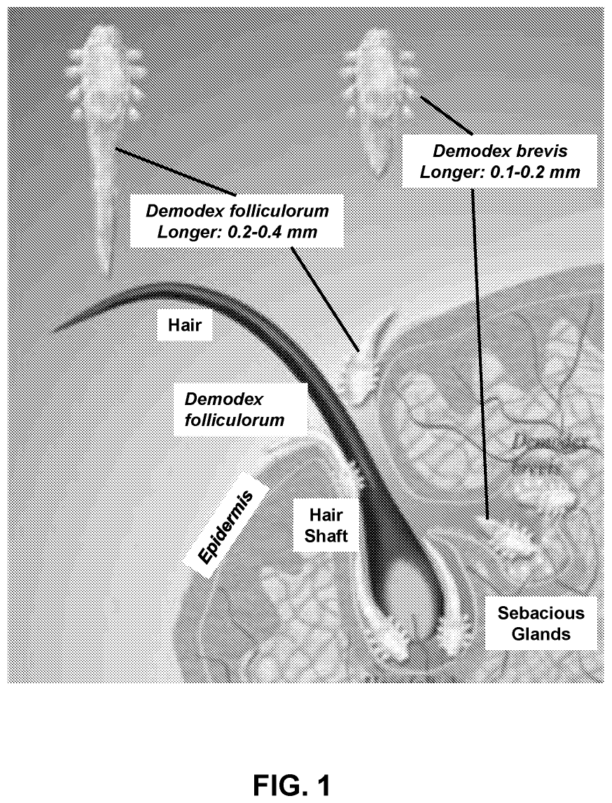

FIG. 2 illustrates Demodex mites in the eye region, including eye lash and meibomian glands. FIG. 2 is adapted from Crystal D. (2016) Know your enemy: rich snippet on demodex. etCETera. 2(1): 12-17.

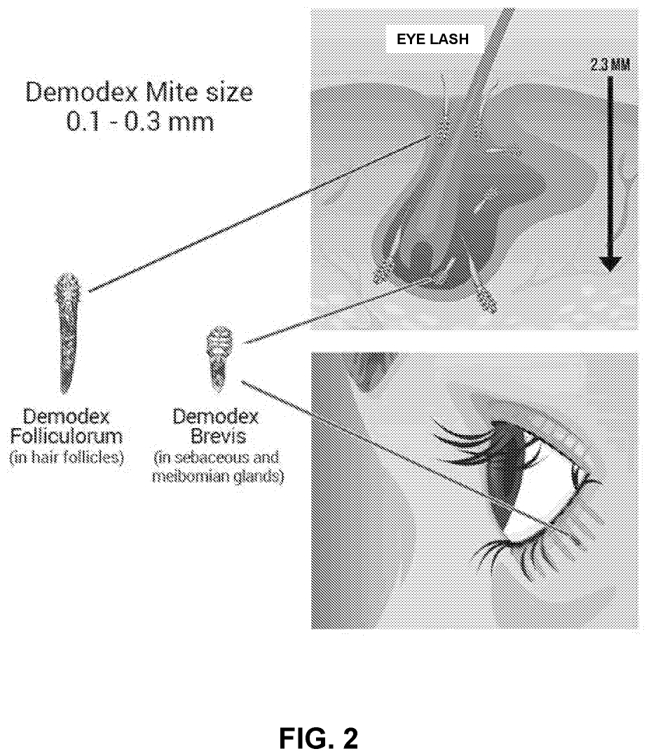

FIG. 3 is a table summary of Demodex survival time for various active agents.

FIG. 4 summarizes the activity of 10% chloroquine in water relative to a water control on activity against Demodex mites in an in vitro assay, with an LT₅₀ of 31.6 hours for chloroquine compared to 44.8 hours for the water control, illustrating the inactivation effect of chloroquine on Demodex.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of the invention.

“Treating” refers to a therapeutic application of chloroquine and/or hydroxychloroquine to at least decrease adverse symptoms caused by the presence of Demodex bevis and/or folliculorum mites. As described herein, there are various observable symptoms, referred herein as “clinical symptoms”, associated with the mites, particularly on the face. The treatment or treating methods described herein may alleviate or at least substantially attenuate any one or more of those clinical symptoms. Categories of those symptoms may not be directly caused by the mite, but instead may arise indirectly due to the presence of mites, and may include an immunological response such as an allergic response and/or a vasomotor response. For example, a response to mites may include a dilation of surface blood vessels to increase blood flow to the region of the face from which an immune signal is generated, thereby resulting in an observable appearance of flushing or redness of the skin. Similarly, an overly robust immune response can lead to typical symptoms related to immunological responses, including inflammation, itching, irritation, scarring, and the like. Other physical manifestations of those symptoms may further include acne and the like. The mites themselves may not directly cause the clinical symptom, but instead may trigger an immune response which then causes the clinical symptom. Similarly, a bacteria or other pathogen carried by the mite may cause the clinical symptom and the treatment that inactivates mite populations may alleviate the clinical symptom after a certain time post-application.

“Clinical symptom”, therefore, refers to a physical, detectable, measurable or observable adverse or unwanted condition associated with the mite-responsible skin affliction. In an aspect, it is a skin condition that is observable. The clinical symptom may further be assessed by the individual, such as by a feeling of pain, burning sensation, itching, or other discomfort expressed by the individual. The methods provided herein are useful for attenuating, alleviating or cessation any such clinical symptoms.

“Attenuating” or “ameliorating” refers to a measurable or quantifiable reduction in the clinical symptom. For example, the individual can indicate whether the feeling of discomfort is reduced. Alternatively or in addition, the reduction may be measured, such as by a reduction in skin discoloration, scarring, or other skin abnormality. As desired, the attenuation may have a quantifiable decrease, such as a decrease of at least 50% compared to pre-treatment. An initial attenuation may then be followed by cessation of the clinical symptom. “Cessation” refers to essentially a disappearance of the clinical symptom such that there is no longer an observable symptom and/or the individual does not have a feeling of discomfort otherwise associate with the skin affliction.

The dosing for the various administration routes may be selected so as to ensure there is sufficient Demodex inactivation, including a portion of or substantially all Demodex mites. In this context, “a portion of” refers to greater than 50% and “substantially all” refers to at least 85%, at least 90%, at least 95%, at least 99.5% or about 99.9% inactivation of total number of mites present.

As used herein, the term mite refers to a Demodex mite, and more particularly for some embodiments a Demodex brevis (d. brevis) mite and/or a Demodex folliculorum (d. folliculorum) mite found in or on the skin of an individual. Because these mites are found in humans, an aspect of any of the treatment methods provided herein is for an individual that is a human.

As outlined herein, Demodex folliculorum and Demodex brevis mites play a role in the rosacea condition. An increased Demodex population has been observed in rosacea patients. For most people, Demodex mites live harmlessly in the skin as a result of either down-regulating host immunity or simply dodging host immune defenses. There is vociferous debate within the dermatology community as to whether or not they are the causative agents of such skin diseases as rosacea and blepharitis (inflammation of the eyelids) a common issue seen in rosacea patients.

Human beings are the one and only host of this ubiquitous mite [2]. In fact, these two mites are considered to be the most common ectoparasite of humans [5]. Women tend to have a higher rate of Demodex infections [4]. The rate of infestation also seems to be correlated with age, with 84% of people at age 60 harboring mites and increasing to 100% in those 70 years and older [6]. Whether those that are immunocompromised are more susceptible to higher infestation rates is unknown, though some studies indicate that AIDs and leukemia patients may be more prone to greater than average numbers [4].

The mites are most commonly found in the scalp, face and upper chest area, with Demodex folliculorum exhibiting a predilection for the hair follicles and Demodex brevis for the sebaceous ducts and meibomian glands at the rim of the eyelids (the sebaceous ducts transfer the waxy sebum that lubricates the skin and hair from the sebum glands; the meibonmian glands are a special type of such gland) [3][4]. Demodex folliculorum are a communal bunch, tending to congregate in the follicle area of the hair or eyelashes with their posterior ends protruding from the follicular pores. Demodex brevis, on the other hand, tend to be more solitary and will occupy the sebaceous glands singly [5]. Both species are tiny, less than 0.4 mm, with elongated, clear bodies and four pairs of stout legs. Demodex brevis is usually a tad shorter, ˜0.1 mm, than Demodex folliculorum. They both have ridged scales along their cephalothorax and sharp, piercing teeth [5].

Short-lived creatures, a mite's life cycle from egg to larva to adult typically lasts on the order of weeks depending on environmental conditions, such as from about 14-24 days, or from 14-18 days. Adults emerge from the follicles and ducts to reproduce at the surface of the skin where females will then deposit eggs in the sebaceous glands. Larva will mature via two nymphal stages in the glands until entering the follicles and ducts as adults to begin the cycle anew [5]. It is hypothesized that both species of mites feed upon sebum as a primary food source but may also feed on follicular and glandular epithelia. The mites are sometimes characterized as obligate ectoparasites, incapable of living outside their human host.

Studies indicate a greater than average mite density, such as greater than five mites per cm², play a role in these two diseases for patients [5]. Research suggests that blockage of the hair follicles and sebaceous ducts by mites may result in epithelial hyperplasia, elicit a phagocytic, granulomatous reaction or bring an inflammatory response due to their waste products [5]. The fact that treatment with certain antibiotics can reduce the severity of rosacea suggests a microbial component to mite-related diseases. For example, researchers isolated from Demodex folliculorum a bacterium Bacillus oleronium that provoked inflammatory responses in 73% of rosacea patients but only 29% of controls [20]. These results suggest that patients with rosacea are sensitized to the bacteria and may be immunologically sensitive to the mites, bacteria or both [20].

Two antigenic proteins found on the bacterium's cell surface in particular appear responsible for the inflammatory response by stimulating peripheral blood mononuclear cell proliferation; one 83 kDa protein showed similarity with heat-shock proteins while the other 62 kDa protein shared amino acid sequence homology with a protease enzyme found to be involved signal transduction as well as carbohydrate metabolism [20]. Additional indication of the pathogenic role of B. oleronius in rosacea may also be found in the sensitivity of the bacterium to many antibiotics shown to be effective in the treatment of rosacea, specifically tetracycline, doxycycline and minocycline [20].

Examples of classic rosacea symptoms include: persistent redness, flushing especially with common rosacea triggers (cosmetics, stress, alcohol, heat, sun exposure, exercise, spicy foods), telangiectasias on the nose and cheeks, bumps and pimples, dry appearance, tight or swollen skin, burning and itching skin.

According to the national Rosacea Society seborrheic dermatitis may be the most common skin condition to occur at the same time as rosacea. Although the two disorders are thought to be unrelated, a recent clinical study found that 26 percent of patients with rosacea also had facial seborrheic dermatitis and 28 percent had seborrheic dermatitis of the scalp. Additionally, a survey by the National Rosacea Society of 1,099 rosacea patients found that 25 percent had also been diagnosed with this condition [1].

A study identified a bacillus bacterium inside Demodex mites, where the bacteria releases two proteins that trigger an inflammation in patients with facial rosacea.” [37]. At least one type of bacteria is associated with Demodex mites and rosacea. This bacteria is Bacillus oleronius according to an NRS press release [38] (“This indicates that the Bacillus bacteria found in the Demodex mite produce an antigen that could be responsible for the tissue inflammation associated with papulopustular rosacea.”) Other studies state, “Antigenic proteins related to a bacterium (B. oleronius), isolated from a Demodex folliculorum mite, have the potential to stimulate an inflammatory response in patients with papulopustular rosacea.” [39]; and “The strong correlation provides a better understanding of comorbidity between Demodex mites and their symbiotic B. oleronius in facial rosacea and blepharitis.” [40]

Potential causes of rosacea include increased facial bloodflow, altered response patterns of facial bloodflow, photo damage, oxygen free radical, UV light exposure, heat exposure, the proliferation of Demodex mites, Helicobacter pylori infection, differences in bacterial proteins found in association with inflammatory lesions of rosacea, and the proinflammatory bacterium Bacillus oleronius found in the gut of the Demodex mites more commonly in rosacea-affected facial skin.[64] Due to limited basic science research on rosacea, the validity and/or magnitude of the role of these potential causes in the pathogenesis of rosacea has been debated.[64]

The pathophysiologic mechanisms of rosacea are difficult to research because there are major pitfalls in laboratory techniques used to study the disease. With gene array expression analysis as a rough indicator of change in mRNA levels of multiple genes, results may or may not correlate with a protein of interest. Similarly, RT-PCR a more specific indicator of changes in mRNA, but results may or may not correlate with production or activity of a protein of interest. Using Immunohistochemistry the immunoreactivity may not be uniform within a specimen and data presented may be subject to selection bias or lack of specificity depending on the quality of the antibody. Zymography results depend on proper incubation and digestion techniques. No robust animal models exist for rosacea; assays used in animals are surrogates for some putative aspects of rosacea pathophysiology. Finally, cell culture is difficult because multiple cell types within and surrounding the follicle are involved in rosacea; interactions between cell types cannot be modeled with current cell culture techniques.[64]

Demodex folliculorum and Demodex brevis are cosmopolitan, obligatory parasites. Epidemiological studies have established a clear association between these species and various facial diseases in humans. However, not much is known of the ecology of these mites. One reason for this is because it is difficult to culture the mites. [65] There are few studies on the ecology of Demodex folliculorum and Demodex brevis, and maintenance in vitro has not been successfully achieved. Empirical studies lack large numbers of standard Demodex folliculorum and Demodex brevis, which critically restricts further study of their pathogenicity. For a long time, the only means to obtain Demodex folliculorum and Demodex brevis samples was to conduct a census using the cellophane tape method, which is time-consuming and labor-intensive. In addition, the Demodex folliculorum and Demodex brevis obtained in this manner may not meet the requirements for standard experiments and cannot be kept for long due to their aptness to die. [66] Because animal and in vitro models are not available for Demodex Folliculorum and Demodex brevis mites, with the mites tending to die off after about 60 to 80 hours at most, basic research with the mites is difficult and not practical.

The fundamental understanding that Demodex may be a systemic cause of rosacea indicates a natural treatment with a known miticide, such as tea tree oil [43], may assist with symptom alleviation. Tea tree oil may be applied using several formulations, including 100%. The tea tree oil assists with blepharitis but is not well tolerated by skin, especially in higher concentrations. The redness, scaling and erythema associated with demodatic skin afflictions will persist post treatment even with 100% tea tree oil. To better understand the efficacy of the treatments provided herein, it is useful to understand the current state of the art with respect to other treatments.

### Example 1: Chloroquine and/or Hydroxychloroquines

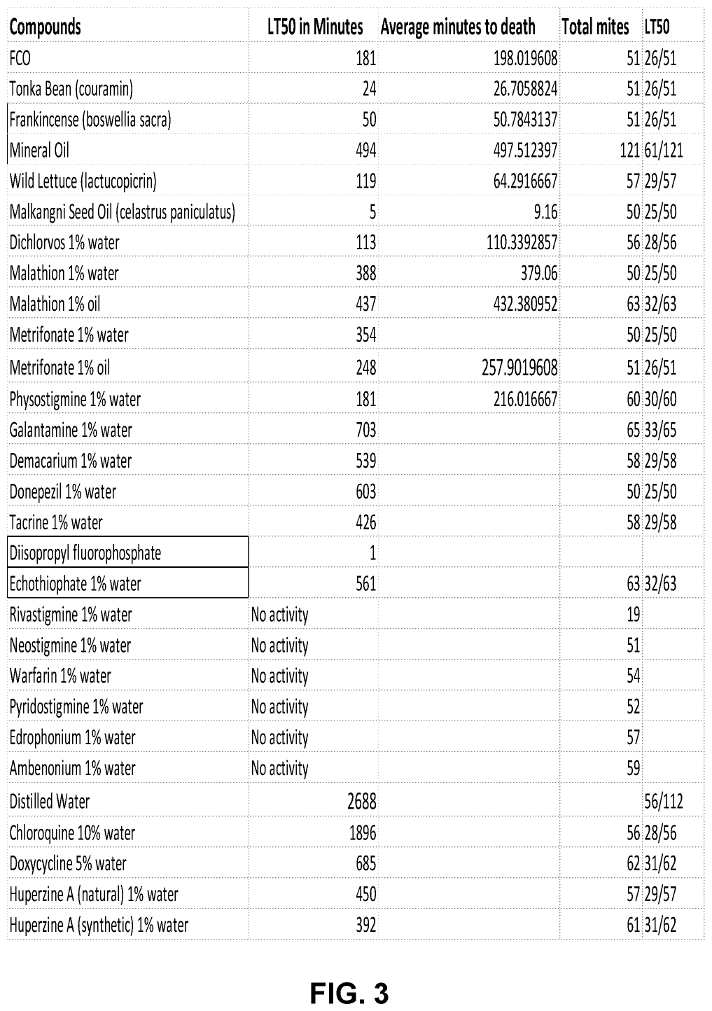

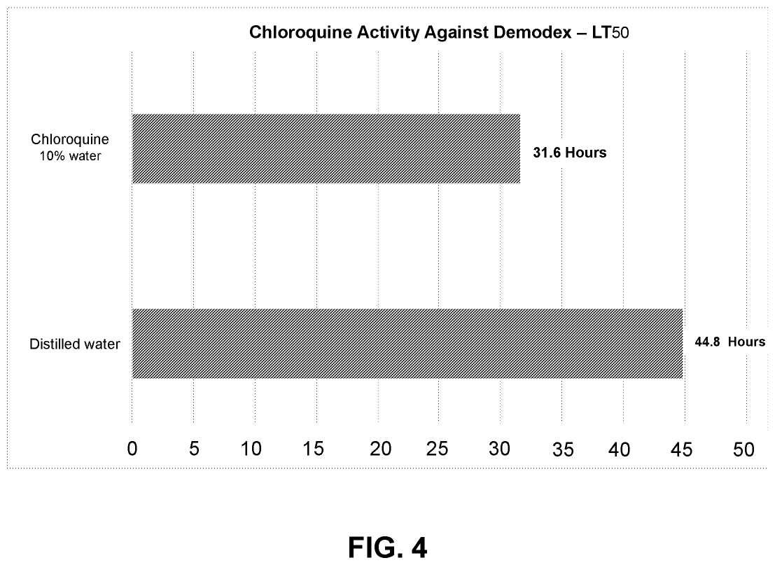

An in vitro assay establishes that the anti-malarial drug chloroquine has activity against Demodex mites, (FIG. 3) and (FIG. 4).

In an embodiment of this invention, chloroquine and/or hydroxychloroquine, are administered topically to a patient with an active skin condition in which the underlying cause is a Demodex mite, such as a Demodex brevis and/or Demodex folliculorum mite. Because the target organisms, Demodex brevis and Demodex folliculorum, are ectoparasites in the mite family, an effective treatment must be capable of inactivating or eradicating substantially the entire lifecycle of such a microscopic insect, including egg, larval, and adult stages, for long-term efficacy. For this reason, this embodiment treats such patients with several doses. Such spacing allows time for Demodex eggs that may not inactivate as a result of the chloroquine and/or hydroxychloroquine application, to hatch into immature mites that are killed before they can mature into egg-producing adults. After the chloroquine and/or hydroxychloroquine carries out its miticidal activity on skin Demodex brevis and Demodex folliculorum organisms, inflammatory responses to them begin to diminish but remnants of the dead mites still elicit responses observed as clinical symptoms, such as some flushing and lesion formation, until the cleanup processes of the body remove them, a process requiring six to eight weeks. During this initial phase of chloroquine and/or hydroxychloroquine administration, conventional anti-rosacea medications such as oral tetracycline and topical metronidazole can optionally be employed to suppress early flareups and to provide early clinical improvement and response. No such medications are needed to treat manifestations of rosacea after six to eight weeks have elapsed. After prolonged intervals of freedom from rosacea symptoms, should classic signs begin to reappear, treatment can be repeated. The chloroquine and/or hydroxychloroquine is formulated into a cosmetically-acceptable topical lotion, cream, shampoo, or gel and applied especially to skin affected by rosacea and any area possibly inhabited by Demodex brevis and Demodex folliculorum. Because of the barrier effect the skin presents to the penetration of topical medications, such a route of treatment with chloroquine and/or hydroxychloroquine is anticipated to require once or twice daily applications for as long as six weeks to achieve sufficient follicle penetration and effective miticidal activity. A topical formulation that could achieve this effect may contain 5% or less of the chloroquine and/or hydroxychloroquines. Lower percentages of chloroquine and/or hydroxychloroquine that retains sufficient miticidal effect and successful skin condition treatment is preferred so as to limit or minimize potential side effects of the chloroquine and/or hydroxychloroquine. Further, full body treatment is optionally useful for preventing reintroduction of the mites onto skin, such as facial skin, from other body locations that may not present clinical symptoms.

### Example 2: Demodex and Rosacea

The etiology of rosacea is still not fully understood, although many theories have been advanced. It has been a frequently discussed topic in medical circles but a full consensus has not ever been reached. The prominent presence of erythema (redness) and flushing of the face of affected persons with aggravation from heat, sunshine, and alcohol has focused attention on this aspect of the disease. The newest and most common hypothesis is based on the characteristic presence of the parasite Demodex folliculorum in the case of patients suffering from rosacea. This organism is absent in the other forms of acne such as common acne. Other factors have been described as possibly contributing towards the development of rosacea, such as hormonal factors and especially endocrine factors, climatic and immunological factors, and bacterial factors via the presence of Helicobacter pylori, a bacterium associated with gastrointestinal disorders.

Treatment with medications to block such vasomotor flushing have no effect on other aspects of the disease such as papules and pustules. Treatment with oral and topical antibiotics has been shown to effectively block progression of rosacea through a poorly understood anti-inflammatory mechanism or by destroying bacteria associated Demodex folliculorum mites. Antibiotics have to be continually administered and are in many cases only marginally effective. Many times patients cannot tolerate the side effects related to the oral antibiotics.

Although hypothesized as a root cause of rosacea, many rosacea subtypes and seborheic dermatitis, Demodex brevis and Demodex folliculorum there is not consensus regarding the root cause and no commercially viable pharmacological solutions are available for treating Demodex brevis and Demodex folliculorum. Democodosis presents like rosacea or seborrheic dermatitis but is confirmed as being caused by Demodex mites. Reaction to the presence or metabolic activity of Demodex mites in facial follicles has long been discussed as a cause of rosacea but previous studies where topical miticides other than chloroquine and/or hydroxychloroquines have been used have shown inconsistent and marginal results.

A study has found that the bacterium Bacillus oleronius stimulates an immune system response, inducing high levels of T-cell proliferation, in 79 percent of patients with subtype 2 rosacea, compared with only 29 percent of patients without the disorder. T-cell proliferation induces an inflammatory response, evident as papules and pustules. This suggests that the Bacillus bacteria found in the Demodex mite produces an antigen that could be responsible for the tissue inflammation associated with papulopustular rosacea. Many current antibiotic treatments for rosacea are theorized to be effective based on their ability to effectively combat Bacillus oleronius.

Demodectic rosacea is without question a variant of rosacea when treatment for Demodex mites improves rosacea. But most rosacea patients are never treated for Demodex mite and, hence they cannot be ruled out of a rosacea case unless a rosacea patient is treated for Demodex and does not respond. There is much controversy when a discussion about demodectic rosacea is introduced. While some assert this a theory, the facts are that there are more clinical reports on Demodex mites and rosacea than any other topic (other than prescription drug treatment). [13] It is an established fact that demodectic rosacea happens since in some cases treatment for Demodex improves rosacea. Therefore, it is important to rule out demodectic rosacea in a differential diagnosis if you have a red face. One report characterizes it may be a ‘missing link’ in the understanding of rosacea. [41]

Another report states, “Because Demodex mites are ubiquitous, their potential as human pathogens has often been ignored. This contribution focuses on the growing body of evidence linking Demodex mites with various skin disorders. Histologically, spongiosis and lymphoid inflammation are regularly seen in follicles containing Demodex mites. In animals, they are well established as a cause of mange, and a human counterpart-demodectic alopecia-appears to exist. There is also a statistical association between Demodex mite density and rosacea, facial itching, and chronic blepharitis. Papulovesicular rosacea like lesions and spiny blepharitis often respond to agents that reduce Demodex numbers. Although these observations are not sufficient to fulfill Koch's postulates, Koch's postulates are also not fulfilled for the association between brown recluse spiders and dermal necrosis or the association between streptococci and guttate psoriasis. The evidence linking Demodex mites to human disease has implications regarding treatment.” [12]

While there is doubt regarding demodex's role in rosacea (some characterize Demodex as an ‘innocent by-stander’) there is growing evidence that demodectic rosacea should be ruled out in every case of rosacea.

Demodex and its connection with rosacea is a heavily researched and reported topic. Other relevant topics include clinical reports on metronidazole or other prescription treatments for rosacea. [13] Demodex continues to be debated not only by rosaceans but also in the medical community. Some characterize this issue as Demodex mites being incidental parasites that prey on compromised skin causing secondary symptoms, not unlike bacteria and fungi. Based on this characterization, their opinion is that the Demodex mites are not the primary cause of rosacea and that not all rosaceans have Demodex as a relevant factor. Clinicians and researchers further characterize Demodex folliculorum as being mentioned as an aggravating factor to rosaceans for many decades and yet, but that no formal double blind studies have addressed this topic. Generally the debate centers on whether Demodex plays an active role in rosacea or is passive, with the issue being distilled down to which comes first, the rosacea or demodex. If Demodex is the cause of rosacea, than why current miticides do not work is also a reason the Demodex theory is dismissed by many dermatologists. See, e.g., [14] (“Rosacea experts all agree that this mite plays no real role in the development of progression of rosacea.”); [15] (“I have always pushed the line that Demodex mites have thus far only been proven to be innocent bystanders in rosacea symptoms.”); and [43] (“The status of Demodex folliculorum and its role in rosacea is still an open area of study. It has been difficult to prove that there is or is not a link between the mite and rosacea. In my mind, Demodex mites remain as an innocent bystander.”). In contrast, see [42] (“We do need more research. Demodex have been the subject of an enormous amount of rosacea research, so it pains me to say this!” (describing a study by [Forton FM] which is described in [42] as “in the pure speculation category.”)); [59] (titled, “More Demodex Dreaming: Mites are the Chicken?”); and [60] (referring to an article published by the NRS, “The Chicken, not the Egg?”).

From the above examples, the medical community as well as others view demodectic rosacea as a passive player in rosacea, but there is conflicting evidence.

The methods provided herein recognize demodectic rosacea as an established fact that should be included in any differential diagnosis of rosacea. More and more reports confirm the need to rule out demodectic rosacea. Other possible causes of facial redness include SIBO, hypertension, hyperthyroidism, carciniods, adult acne, allergic reaction and lupus.

Because intense pulsed light (IPL) kills mites [45], in an aspect any of the methods provided herein may further comprise administration of an chloroquine and/or hydroxychloroquine along with co- or post-administration of light, including intense light or intense pulsed light.

Testing for Demodectic rosacea—Demodex Density Counts: Techniques are available to non-invasively detect, image and quantify Demodex mites in facial skin of patients with rosacea, including confocal laser scanning microscopy. See, e.g., [54] (“With the help of CLSM it is possible to non-invasively detect, image and quantify Demodex mites in facial skin of patients with rosacea.”). There are, however, “limitations to the use of this method to accurately detect absolute numbers of mites in human skin.” [61]

Two other tests include density count and empirical tests by applying a cream like permethrin or crotamiton daily for 2-3 weeks and see if anything unusual happens. The first test, counting mite densities, is not too helpful. A person may have many mites or only a few, but the density test provides no indication if you have a problem with the demodex. The test merely counts mites in a random column of extracted skin. A nice number can be produced for a graph for some research papers. The major issue here is a physician may deny treatment if the number does not pass some arbitrary threshold. The second test, the empirical test, is more helpful. If something unusual and significant happens when applying the cream, like a sudden improvement or worsening, then the problem is likely to be linked to the death of demodex. If nothing happens, then Demodex is not a problem and can be excluded.

Papulopustular rosacea (PPR) is similar to demodectic rosacea. It may be an allergy to Demodex or to a bacteria associated with demodex. Some people are allergic, others are not. Unlike other common allergies, this allergen is stuck in the skin as you cannot just choose to avoid demodex. It becomes necessary to kill all the mites to bring relief or to suppress the symptoms with a perpetual course of antibiotics. The symptoms of PPR, the red skin, dry skin, blepharitis, and the relentless onslaught of mosquito bite-like papules that sting/tickle are classic allergy symptoms. Once all the mites are dead and are out of the skin, these symptoms stop and the skin returns to normal.

### Example 3: Administration and Formulation

Salts and Prodrugs: The invention contemplates pharmaceutically active compounds either chemically synthesized or formed by in vivo biotransformation to compounds set forth herein.

Compounds of this invention and compounds useful in the methods of this invention include those of the compounds and formula(s) described herein and pharmaceutically-acceptable salts and esters of those compounds. In embodiments, salts include any salts derived from the acids and bases of the formulas herein which are acceptable for use in human or veterinary applications. In embodiments, the term ester refers to hydrolyzable esters of compounds of the names and formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

Compounds of the invention and used in the methods of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in: Design of Prodrugs, edited by H. Bundgaard, (Elsevier, 1985); Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191 (1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug, can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in: T. Higuchi and V. Stella, Pro-drugs as Novel Delivery Systems, V. 14 of the A.C.S. Symposium Series; and in Edward B. Roche, ed., Bioreversible Carriers in Drug Design, American Pharmaceutical Association and Pergamon Press (1987).

The active agent may also comprise quinine, such as having the following chemical structure, and salts thereof:

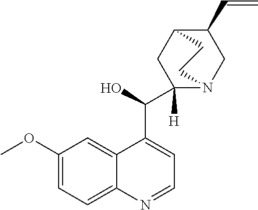 [SMILES: C=C[C@H]1C[N@]2CC[C@H]1C[C@H]2[C@H](O)c1ccnc2ccc(OC)cc12]

Quinine may be provided as a salt. It may also be provided in various related formulations, including the hydrochloride, dihydrochloride, sulfate, bisulfate and gluconate. Quinine salts may be given orally or intravenously (IV); quinine gluconate may also be given intramuscularly (IM) or rectally (PR).

Active ingredients of the invention can be formulated with pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include, among others, halides (e.g., F⁻, Cl⁻, Br⁻, At⁻), sulfate, acetates (e.g., acetate, trifluoroacetate), ascorbates, aspartates, benzoates, citrates, and lactate.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts can be derived from amino acids, including, but not limited to, cysteine. Other pharmaceutically acceptable salts can be found, for example, in Stahl et al., Handbook of Pharmaceutical Salts: Properties, Selection, and Use, Wiley-VCH, Verlag Helvetica Chimica Acta, Zurich, 2002. (ISBN 3-906390-26-8).

5.b: Efficacy: Typically, a compound of the invention, or pharmaceutically acceptable salt thereof, is administered to a subject in a diagnostically or therapeutically effective amount.

Compositions for oral administration can be, for example, prepared in a manner such that a single dose in one or more oral preparations contains at least about 20 mg of the present compound per square meter of subject body surface area, or at least about 50, 100, 150, 200, 300, 400, or 500 mg of the present compound per square meter of subject body surface area (the average body surface area for a human is, for example, 1.8 square meters). In particular, a single dose of a composition for oral administration can contain from about 20 to about 600 mg, and in certain aspects from about 20 to about 400 mg, in another aspect from about 20 to about 300 mg, and in yet another aspect from about 20 to about 200 mg of the present compound per square meter of subject body surface area. Compositions for parenteral administration can be prepared in a manner such that a single dose contains at least about 20 mg of the present compound per square meter of subject body surface area, or at least about 40, 50, 100, 150, 200, 300, 400, or 500 mg of the present compound per square meter of subject body surface area. In particular, a single dose in one or more parenteral preparations contains from about 20 to about 500 mg, and in certain aspects from about 20 to about 400 mg, and in another aspect from about 20 to about 450 mg, and in yet another aspect from about 20 to about 350 mg of the present compound per square meter of subject body surface area. It should be recognized that these oral and parenteral dosage ranges represent generally preferred dosage ranges, and are not intended to limit the invention. The dosage regimen actually employed can vary widely, and, therefore, can deviate from the generally preferred dosage regimen. It is contemplated that one skilled in the art will tailor these ranges to the individual subject.

Toxicity and therapeutic efficacy of such compounds and bioconjugates can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compounds and bioconjugates that exhibit large therapeutic indices are preferred. While compounds and bioconjugates exhibiting toxic side effects can be used, care should be taken to design a delivery system that targets such compounds and bioconjugates to the site affected by the disease or disorder in order to minimize potential damage to unaffected cells and reduce side effects.

Data obtained from the cell culture assays and animal studies can be used in formulating a range of dosages for use in humans and other mammals. The dosage of such compounds and bioconjugates lies preferably within a range of circulating plasma or other bodily fluid concentrations that include the ED₅₀ and provides clinically efficacious results (i.e., reduction in disease symptoms). The dosage can vary within this range depending upon the dosage form employed and the route of administration utilized. For any compound and bioconjugate of the present invention, the therapeutically effective amount can be estimated initially from cell culture assays. A dosage can be formulated in animal models to achieve a circulating plasma concentration range that includes the ED₅₀ (the concentration of the test compound that achieves a half-maximal inhibition of symptoms) as determined in cell culture. Such information can be used to more accurately determine useful dosages in humans and other mammals. Compound and bioconjugate levels in plasma can be measured, for example, by high performance liquid chromatography.

An amount of a compound or bioconjugate that can be combined with a pharmaceutically acceptable carrier to produce a single dosage form will vary depending upon the patient treated and the particular mode of administration. It will be appreciated by those skilled in the art that the unit content of a compound/bioconjugate contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage and dosage regime for treating a disease or condition can be selected in accordance with a variety of factors, including the type, age, weight, sex, diet and/or medical condition of the patient, the route of administration, pharmacological considerations such as activity, efficacy, pharmacokinetic and/or toxicology profiles of the particular compound/bioconjugate employed, whether a compound/bioconjugate delivery system is utilized, and/or whether the compound/bioconjugate is administered as a pro-drug or part of a drug combination. Thus, the dosage regime actually employed can vary widely from subject to subject, or disease to disease and different routes of administration can be employed in different clinical settings.

The identified compounds/bioconjugates monitor, treat, inhibit, control and/or prevent, or at least partially arrest or partially prevent, diseases and conditions of interest and can be administered to a subject at therapeutically effective amounts and optionally diagnostically effective amounts. Compositions/formulations of the present invention comprise a therapeutically effective amount (which can optionally include a diagnostically effective amount) of at least one compound or bioconjugate of the present invention. Subjects receiving treatment that includes a compound/bioconjugate of the invention are preferably animals (e.g., mammals, reptiles and/or avian), more preferably humans, horses, cows, dogs, cats, sheep, pigs, and/or chickens, and most preferably humans.

5.c: Administration: The preferred composition depends on the route of administration. Any route of administration can be used as long as the target of the compound or pharmaceutically acceptable salt is available via that route. Suitable routes of administration include, for example, oral, intravenous, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

In an embodiment, the invention provides a method for treating a medical condition comprising administering to a subject (e.g. patient) in need thereof, a therapeutically effective amount of a composition of the invention, such as an chloroquine and/or hydroxychloroquine composition. In an embodiment, the invention provides a method for diagnosing or aiding in the diagnosis of a medical condition comprising administering to a subject in need thereof, a diagnostically effective amount of a composition of the invention. In an embodiment, the medical condition is a skin condition or dermatological diseases.

The diagnostic and therapeutic formulations of this invention can be administered alone, but can be administered with a pharmaceutical carrier selected upon the basis of the chosen route of administration and standard pharmaceutical practice.

Any suitable form of administration can be employed in connection with the diagnostic and therapeutic formulations of the invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations can also include aerosol formulation, creams, gels, solutions, etc. The present compositions, preparations and formulations are administered in doses effective to achieve the desired diagnostic and/or therapeutic effect. Such doses can vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations can also optionally include stabilizing agents and skin penetration enhancing agents.

(i) Parenteral Administration:

Compounds and bioconjugates of the present invention can be formulated for parenteral administration by injection (e.g., by bolus injection or continuous infusion). Formulations for injection can be presented in unit dosage form in ampoules or in multi-dose containers with an optional preservative added. The parenteral preparation can be enclosed in ampoules, disposable syringes or multiple dose vials made of glass, plastic or the like. The formulation can take such forms as suspensions, solutions or emulsions in oily or aqueous vehicles, and can contain formulatory agents such as suspending, stabilizing and/or dispersing agents.

For example, a parenteral preparation can be a sterile injectable solution or suspension in a nontoxic parenterally acceptable diluent or solvent (e.g., as a solution in 1,3-butanediol). Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid can be used in the parenteral preparation.

Alternatively, compounds and bioconjugates of the present invention can be formulated in powder form for constitution with a suitable vehicle, such as sterile pyrogen-free water, before use. For example, a compound/bioconjugate suitable for parenteral administration can include a sterile isotonic saline solution containing between 0.1 percent and 90 percent weight per volume of the compound/bioconjugate. By way of example, a solution can contain from about 5 percent to about 20 percent, more preferably from about 5 percent to about 17 percent, more preferably from about 8 to about 14 percent, and still more preferably about 10 percent weight per volume of the compound/bioconjugate. The solution or powder preparation can also include a solubilizing agent and a local anesthetic such as lignocaine to ease pain at the site of the injection. Other methods of parenteral delivery of compounds/bioconjugates will be known to the skilled artisan and are within the scope of the invention.

(ii) Oral Administration:

For oral administration, a compound/bioconjugate of the invention can be formulated to take the form of tablets or capsules prepared by conventional means with one or more pharmaceutically acceptable carriers (e.g., excipients such as binding agents, fillers, lubricants and disintegrants).

(iii) Controlled-Release Administration:

Controlled-release (or sustained-release) preparations can be formulated to extend the activity of a compound/bioconjugate and reduce dosage frequency. Controlled-release preparations can also be used to effect the time of onset of action or other characteristics, such as blood levels of the compound/bioconjugate, and consequently affect the occurrence of side effects.

Controlled-release preparations can be designed to initially release an amount of a compound/bioconjugate that produces the desired therapeutic effect, and gradually and continually release other amounts of the compound/bioconjugate to maintain the level of therapeutic effect over an extended period of time. In order to maintain a near-constant level of a compound/bioconjugate in the body, the compound/bioconjugate can be released from the dosage form at a rate that will replace the amount of compound/bioconjugate being metabolized and/or excreted from the body. The controlled-release of a compound/bioconjugate can be stimulated by various inducers, e.g., change in pH, change in temperature, enzymes, water, and/or other physiological conditions or molecules.

Controlled-release systems can include, for example, an infusion pump which can be used to administer the compound/bioconjugate in a manner similar to that used for delivering insulin or chemotherapy to the body generally, or to specific organs or tumors. Typically, using such a system, the compound/bioconjugate is administered in combination with a biodegradable, biocompatible polymeric implant that releases the compound/bioconjugate over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target (e.g., organ, tissue, or group of cells), thus requiring only a fraction of a systemic dosage.

Compounds/bioconjugates of the invention can be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compounds/bioconjugates will be known to the skilled artisan and are within the scope of the invention.

(iv) Inhalation Administration:

Compounds/bioconjugates of the invention can be administered directly to the lung of a patient/subject by inhalation. For administration by inhalation, a compound/bioconjugate can be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas can be used to deliver a compound/bioconjugate directly to the lung. MDI devices are available from a number of suppliers such as 3M Corporation, Aventis, Boehringer Ingleheim, Forest Laboratories, GlaxoSmithKline, Merck & Co. and Vectura.

Alternatively, a Dry Powder Inhaler (DPI) device can be used to administer a compound/bioconjugate to the lung. DPI devices typically use a mechanism such as a burst of gas to create a cloud of dry powder inside a container, which can then be inhaled by the patient. DPI devices are also well known in the art and can be purchased from a number of vendors which include, for example, GlaxoSmithKline, Nektar Therapeutics, Innovata and Vectura. A popular variation is the multiple dose DPI (“MDDPI”) system, which allows for the delivery of more than one therapeutic dose. MDDPI devices are available from companies such as AstraZeneca, GlaxoSmithKline, TEVA, Merck & Co., SkyePharma and Vectura. For example, capsules and cartridges of gelatin for use in an inhaler or insufflator can be formulated containing a powder mix of the compound/bioconjugate and a suitable powder base such as lactose or starch for these systems.

Another type of device that can be used to deliver a compound/bioconjugate to the lung is a liquid spray device supplied, for example, by Aradigm Corporation. Liquid spray systems use extremely small nozzle holes to aerosolize liquid compound/bioconjugate formulations that can then be directly inhaled into the lung. For example, a nebulizer device can be used to deliver a compound/bioconjugate to the lung. Nebulizers create aerosols from liquid compound/bioconjugate formulations by using, for example, ultrasonic energy to form fine particles that can be readily inhaled. Examples of nebulizers include devices supplied by Aventis and Battelle.

In another example, an electrohydrodynamic (“EHD”) aerosol device can be used to deliver a compound/bioconjugate to the lung. EHD aerosol devices use electrical energy to aerosolize liquid compound/bioconjugate solutions or suspensions. The electrochemical properties of the compound/bioconjugate formulation are important parameters to optimize when delivering this compound/bioconjugate to the lung with an EHD aerosol device. Such optimization is routinely performed by one of skill in the art. Other methods of intra-pulmonary delivery of compounds/bioconjugates will be known to the skilled artisan and are within the scope of the invention.

Liquid compound/bioconjugate formulations suitable for use with nebulizers and liquid spray devices and EHD aerosol devices will typically include the compound/bioconjugate with a pharmaceutically acceptable carrier. In one exemplary embodiment, the pharmaceutically acceptable carrier is a liquid such as alcohol, water, polyethylene glycol or a perfluorocarbon. Optionally, another material can be added to alter the aerosol properties of the solution or suspension of the compound/bioconjugate. For example, this material can be a liquid such as an alcohol, glycol, polyglycol or a fatty acid. Other methods of formulating liquid compound/bioconjugate solutions or suspensions suitable for use in aerosol devices are known to those of skill in the art.

(v) Depot Administration:

A compound/bioconjugate of the invention can be formulated as a depot preparation. Such long-acting formulations can be administered by implantation (e.g., subcutaneously or intramuscularly) or by intramuscular injection. Accordingly, the compound/bioconjugate can be formulated with suitable polymeric or hydrophobic materials such as an emulsion in an acceptable oil or ion exchange resin, or as sparingly soluble derivatives such as a sparingly soluble salt. Other methods of depot delivery of compounds/bioconjugates will be known to the skilled artisan and are within the scope of the invention.

(vi) Topical Administration:

For topical application, a compound/bioconjugate can be combined with a pharmaceutically acceptable carrier so that an effective dosage is delivered, based on the desired activity ranging from an effective dosage, for example, of 0.1 μM to 20 M, and any sub-ranges thereof. In one aspect of the invention, a topical formulation of a compound/bioconjugate can be applied to the skin. The pharmaceutically acceptable carrier can be in the form of, for example, and not by way of limitation, a body wash, shampoo, ointment, cream, gel, paste, foam, aerosol, suppository, pad or gelled stick.

A topical formulation can include a therapeutically effective amount of a compound/bioconjugate in an ophthalmologically acceptable excipient such as buffered saline, mineral oil, vegetable oils such as corn or arachis oil, petroleum jelly, Miglyol 182, alcohol solutions, or liposomes or liposome-like products. Any of these formulations of such compounds/bioconjugates can include preservatives, antioxidants, antibiotics, immunosuppressants, and other biologically or pharmaceutically effective agents that do not exert a significant detrimental effect on the compound/bioconjugate. Other methods of topical delivery of compounds/bioconjugates will be known to the skilled artisan and are within the scope of the invention. Topical formulations of the invention further include those comprising one or more compositions useful for penetrating the skin, such as dimethyl sulfoxide (DMSO).

(vii) Rectal Administration:

Compounds/bioconjugates of the invention can be formulated in rectal formulations such as suppositories or retention enemas that include conventional suppository bases such as cocoa butter or other glycerides and/or binders and/or carriers such as triglycerides, microcrystalline cellulose, gum tragacanth or gelatin. Rectal formulations can contain a compound/bioconjugate in the range of 0.5% to 10% by weight, for example. Other methods of rectal delivery of compounds/bioconjugates will be known to the skilled artisan and are within the scope of the invention.

(viii) Other Systems of Administration:

Various other delivery systems are known in the art and can be used to administer the compounds/bioconjugates of the invention. Moreover, these and other delivery systems can be combined and/or modified to promote optimization of the administration of compounds/bioconjugates of the present invention. Exemplary formulations that include compounds/bioconjugates of the present invention are described elsewhere herein (the compounds/bioconjugates of the present invention are indicated as the active ingredient, but those of skill in the art will recognize that pro-drugs and compound combinations are also meant to be encompassed by this term).

5.d: Formulation: In an embodiment, the invention provides a medicament which comprises a therapeutically effective amount of one or more compositions of the invention, such as an chloroquine and/or hydroxychloroquine compound. In an embodiment, the invention provides a medicament which comprises a diagnostically effective amount of one or more compositions of the invention. In an embodiment, the invention provides a method for making a medicament for treatment of a condition described herein, such as the treatment of a skin condition or dermatological disease. In an embodiment, the invention provides a method for making a medicament for diagnosis or aiding in the diagnosis of a condition described herein, such as the diagnosis of a skin condition or dermatological disease. In an embodiment, the invention provides the use of one or more compositions set forth herein for the making of a medicament for the treatment of a skin condition or dermatological disease. In an embodiment, the invention provides the use of one or more compositions set forth herein for the treatment of a disease. In an embodiment, the invention provides the use of one or more compositions set forth herein for the diagnosis of a disease. Compositions of the invention include formulations and preparations comprising one or more of the present chloroquine and/or hydroxychloroquines provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

In an embodiment, the invention provides a pharmaceutical formulation having an active ingredient comprising a composition of the invention, such as an chloroquine and/or hydroxychloroquine compound. In an embodiment, the invention provides a method of synthesizing a composition of the invention or a pharmaceutical formulation thereof, such as an chloroquine and/or hydroxychloroquine compound. In an embodiment, a pharmaceutical formulation comprises one or more excipients, carriers, diluents, and/or other components as would be understood in the art. Preferably, the components meet the standards of the National Formulary (“NF”), United States Pharmacopoeia (“USP”; United States Pharmacopeial Convention Inc., Rockville, Md.), or Handbook of Pharmaceutical Manufacturing Formulations (Sarfaraz K. Niazi, all volumes, ISBN: 9780849317521, ISBN 10: 0849317525; CRC Press, 2004). See, e.g., United States Pharmacopeia and National Formulary (USP 30-NF 25), Rockville, Md.: United States Pharmacopeial Convention (2007 and 2008), and each of any earlier editions; The Handbook of Pharmaceutical Excipients, published jointly by the American Pharmacists Association and the Pharmaceutical Press (Pharmaceutical Press (2005) (ISBN-10: 0853696187, ISBN-13: 978-0853696186)); Merck Index, Merck & Co., Rahway, N.J.; and Gilman et al., (eds) (1996); Goodman and Gilman's: The Pharmacological Bases of Therapeutics, 8th Ed., Pergamon Press. In embodiments, the formulation base of the formulations of the invention comprises physiologically acceptable excipients, namely, at least one binder and optionally other physiologically acceptable excipients. Physiologically acceptable excipients are those known to be usable in the pharmaceutical technology sectors and adjacent areas, particularly, those listed in relevant pharmacopeias (e.g. DAB, Ph. Eur., BP, NF, USP), as well as other excipients whose properties do not impair a physiological use.

This invention also is directed, in part, to pharmaceutical compositions including a therapeutically effective amount of a compound or salt of this invention, as well as processes for making such compositions. Such compositions generally include one or more pharmaceutically acceptable carriers (e.g., excipients, vehicles, auxiliaries, adjuvants, diluents) and can include other active ingredients. Formulation of these compositions can be achieved by various methods known in the art. A general discussion of these methods can be found in, for example, Hoover, John E., Remington's Pharmaceutical Sciences (Mack Publishing Co., Easton, Pa.: 1975). See also, Lachman, L., eds., Pharmaceutical Dosage Forms (Marcel Decker, New York, N. Y., 1980).

The diagnostic and therapeutic formulations of this invention and medicaments of this invention can further comprise one or more pharmaceutically acceptable carriers, excipients, buffers, emulsifiers, surfactants, electrolytes or diluents. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985).

Compositions of the invention include formulations and preparations comprising one or more of the present compounds provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

Compounds and bioconjugates of the present invention can be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. An individual compound/bioconjugate can be administered in combination with one or more additional compounds/bioconjugates of the present invention and/or together with other biologically active or biologically inert agents. Such biologically active or inert agents can be in fluid or mechanical communication with the compound(s)/bioconjugate(s) or attached to the compound(s)/bioconjugate(s) by ionic, covalent, Van der Waals, hydrophobic, hydrophilic or other physical forces. It is preferred that administration is localized in a subject, but administration can also be systemic.

Compounds and bioconjugates of the present invention can be formulated by any conventional manner using one or more pharmaceutically acceptable carriers. Thus, the compound(s)/bioconjugate(s) and their pharmaceutically acceptable salts and solvates can be specifically formulated for administration, e.g., by inhalation or insufflation (either through the mouth or the nose) or oral, buccal, parenteral or rectal administration. The compounds/bioconjugates can take the form of charged, neutral and/or other pharmaceutically acceptable salt forms. Examples of pharmaceutically acceptable carriers include, but are not limited to, those described in REMINGTON'S PHARMACEUTICAL SCIENCES (A. R. Gennaro, Ed.), 20th edition, Williams & Wilkins Pa., USA (2000).

Compounds and bioconjugates of the present invention can be formulated in the form of solutions, suspensions, emulsions, tablets, pills, capsules, powders, controlled- or sustained-release formulations and the like. Such formulations will contain a therapeutically effective amount of the compound/bioconjugate, preferably in purified form, together with a suitable amount of carrier so as to provide the form for proper administration to the patient. The formulation should suit the mode of administration.

Pharmaceutically acceptable carriers that can be used in conjunction with the compounds of the invention are well known to those of ordinary skill in the art. Carriers can be selected based on a number of factors including, for example, the particular compound(s) or pharmaceutically acceptable salt(s) used; the compound's concentration, stability, and intended bioavailability; the condition being treated; the subject's age, size, and general condition; the route of administration; etc. A general discussion related to carriers can be found in, for example, J. G. Naim, Remington's Pharmaceutical Science, pp. 1492-1517 (A. Gennaro, ed., Mack Publishing Co., Easton, Pa. (1985)).

Solid dosage forms for oral administration include, for example, capsules, tablets, gel-caps, pills, dragees, troches, powders, granules, and lozenges. In such solid dosage forms, the compounds or pharmaceutically acceptable salts thereof can be combined with one or more pharmaceutically acceptable carriers. The compounds and pharmaceutically acceptable salts thereof can be mixed with carriers including, but not limited to, lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can include buffering agents, such as sodium citrate, or magnesium or calcium carbonate or bicarbonate. Tablets and pills additionally can, for example, include a coating (e.g., an enteric coating) to delay disintegration and absorption. The concentration of the present compounds in a solid oral dosage form can be from about 5 to about 50% for example, and in certain aspects from about 8 to about 40%, and in another aspect from about 10 to about 30% by weight based on the total weight of the composition.

Liquid dosage forms of the compounds of the invention for oral administration include, for example, pharmaceutically acceptable emulsions, solutions, suspensions, syrups, and elixirs containing inert diluents commonly used in the art (e.g., water). Such compositions also can include adjuvants, such as wetting, emulsifying, suspending, flavoring (e.g., sweetening), and/or perfuming agents. The concentration of the present compounds in the liquid dosage form can be from about 0.01 to about 5 mg, and in certain aspects from about 0.01 to about 1 mg, and in another aspect from about 0.01 to about 0.5 mg per ml of the composition. Low concentrations of the compounds of the invention in liquid dosage form can be prepared in the case that the compound is more soluble at low concentrations. Techniques for making oral dosage forms useful in the invention are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

In some aspects of the invention, tablets or powders for oral administration can be prepared by dissolving the compound in a pharmaceutically acceptable solvent capable of dissolving the compound to form a solution and then evaporating when the solution is dried under vacuum. A carrier can also be added to the solution before drying. The resulting solution can be dried under vacuum to form a glass. The glass can then be mixed with a binder to form a powder. This powder can be mixed with fillers or other conventional tableting agents, and then processed to form a tablet. Alternatively, the powder can be added to a liquid carrier to form a solution, emulsion, suspension, or the like.

In some aspects, solutions for oral administration are prepared by dissolving the compound in a pharmaceutically acceptable solvent capable of dissolving the compound to form a solution. An appropriate volume of a carrier is added to the solution while stirring to form a pharmaceutically acceptable solution for oral administration.

In some embodiments, a liposome or micelle can be utilized as a carrier or vehicle for the composition. For example, in some embodiments, the compound can be a part of the lipophilic bilayers or micelle, and the targeting ligand, if present, can be on the external surface of the liposome or micelle. As another example, a targeting ligand can be externally attached to the liposome or micelle after formulation for targeting the liposome or micelle (which contains the chloroquine and/or hydroxychloroquine agents) to the desired tissue, organ, or other site in the body.

Injectable preparations (e.g., sterile injectable aqueous or oleaginous suspensions) can be formulated according to the known art using suitable dispersing, wetting agents, and/or suspending agents. Acceptable vehicles for parenteral use include both aqueous and nonaqueous pharmaceutically-acceptable solvents. Suitable pharmaceutically acceptable aqueous solvents include, for example, water, saline solutions, dextrose solutions (such as DW5), electrolyte solutions, etc.

In one embodiment, the present compounds are formulated as nanoparticles or microparticles. Use of such nanoparticle or microparticle formulations can be beneficial for some applications to enhance delivery, localization, target specificity, administration, etc. of the compound. Potentially useful nanoparticles and microparticles include, but are not limited to, micelles, liposomes, microemulsions, nanoemulsions, vesicles, tubular micelles, cylindrical micelles, bilayers, folded sheets structures, globular aggregates, swollen micelles, inclusion complex, encapsulated droplets, microcapsules, nanocapsules or the like. As will be understood by those having skill in the art, the present compounds can be located inside the nanoparticle or microparticle, within a membrane or wall of the nanoparticle or microparticle, or outside of (but bonded to or otherwise associated with) the nanoparticle or microparticle. The agent formulated in nanoparticles or microparticles can be administered by any of the routes previously described. In a formulation applied topically, the compound is slowly released over time. In an injectable formulation, the liposome, micelle, capsule, etc., circulates in the bloodstream and is delivered to the desired site (e.g., target tissue).

Preparation and loading of nanoparticles and microparticles are well known in the art. As one example, liposomes can be prepared from dipalmitoyl phosphatidylcholine (DPPC) or egg phosphatidylcholine (PC) because this lipid has a low heat transition. Liposomes are made using standard procedures as known to one skilled in the art (e.g., Braun-Falco et al., (Eds.), Griesbach Conference, Liposome Dermatics, Springer-Verlag, Berlin (1992), pp. 69 81; 91 117. Polycaprolactone, poly(glycolic) acid, poly(lactic) acid, polyanhydride or lipids can be formulated as microspheres. As an illustrative example, the present compounds can be mixed with polyvinyl alcohol (PVA), the mixture then dried and coated with ethylene vinyl acetate, then cooled again with PVA. In a liposome, the present compounds can be within one or both lipid bilayers, in the aqueous between the bilayers, or within the center or core. Liposomes can be modified with other molecules and lipids to form a cationic liposome. Liposomes can also be modified with lipids to render their surface more hydrophilic which increases their circulation time in the bloodstream. The thus-modified liposome has been termed a “stealth” liposome, or a long-lived liposome, as described in U.S. Pat. No. 6,258,378, and in Stealth Liposomes, Lasic and Martin (Eds.) 1995 CRC Press, London. Encapsulation methods include detergent dialysis, freeze drying, film forming, injection, as known to one skilled in the art and disclosed in, for example, U.S. Pat. No. 6,406,713. Optionally, the present compositions and methods include a micelle delivery system, for example, involving one or more PEG-based amphiphilic polymers developed for drug delivery including: PEG-poly(ε-caprolactone), PEG-poly(amino acid), PEG-polylactide or PEG-phospholipid constructs; a cross linked poly(acrylic acid) polymer system, a phospholipid-based system and/or block copolymer systems comprising one or more of the following polymer blocks: a poly(lactic acid) polymer block; a poly(propylene glycol) polymer block; a poly(amino acid) polymer block; a poly(ester) polymer block; a poly (ε-caprolactone) polymer block; a poly(ethylene glycol) block, a poly(acrylic acid) block; a polylactide block; a polyester block; a polyamide block; a polyanhydride block; a polyurethane block; a polyimine block; a polyurea block; a polyacetal block; a polysaccharide block; and a polysiloxane block.

Suitable pharmaceutically-acceptable nonaqueous solvents include, but are not limited to, the following (as well as mixtures thereof):

(i) Alcohols (these include, for example, α-glycerol formal, δ-glycerol formal, 1, 3-butyleneglycol, aliphatic or aromatic alcohols having from 2 to about 30 carbons (e.g., methanol, ethanol, propanol, isopropanol, butanol, t-butanol, hexanol, octanol, amylene hydrate, benzyl alcohol, glycerin (glycerol), glycol, hexylene, glycol, tetrahydrofuranyl alcohol, cetyl alcohol, and stealyl alcohol), fatty acid esters of fatty alcohols (e.g., polyalkylene glycols, such as polypropylene glycol and polyethylene glycol), sorbitan, sucrose, and cholesterol);

(ii) Amides, which include, for example, dimethylacetamide (DMA), benzyl benzoate DMA, dimethylformamide, N-hydroxyethyO-lactamide, N, N-dimethylacetamide-amides, 2-pyrrolidinone, 1-methyl-2-pyrrolidinone, and polyvinylpyrrolidone;

(iii) Esters, which include, for example, acetate esters (e.g., monoacetin, diacetin, and triacetin), aliphatic and aromatic esters (e.g., ethyl caprylate or octanoate, alkyl oleate, benzyl benzoate, or benzyl acetate), dimethylsulfoxide (DMSO), esters of glycerin (e.g., mono, di, and tri-glyceryl citrates and tartrates), ethyl benzoate, ethyl acetate, ethyl carbonate, ethyl lactate, ethyl oleate, fatty acid esters of sorbitan, glyceryl monostearate, glyceride esters (e.g., mono, di, or tri-glycerides), fatty acid esters (e.g., isopropyl myristrate), fatty acid derived PEG esters (e.g., PEG-hydroxyoleate and PEG-hydroxystearate), N-methyl pyrrolidinone, pluronic 60, polyoxyethylene sorbitol oleic polyesters (e.g., poly(ethoxylated)₃₀₋₆₀sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate), polyoxyethylene sorbitan esters (e.g., polyoxyethylene-sorbitan monooleate, polyoxyethylene-sorbitan monopalmitate, polyoxyethylene-sorbitan monolaurate, polyoxyethylene-sorbitan monostearate, and POLYSORBATE 20, 40, 60, and 80 (from ICI Americas, Wilmington, Del.)), polyvinylpyrrolidone, alkyleneoxy modified fatty acid esters (e.g., polyoxyl 40 hydrogenated castor oil and polyoxyethylated castor oils, such as CREMOPHOR EL solution or CREMOPHOR RH 40 solution), saccharide fatty acid esters (i.e., the condensation product of a monosaccharide (e.g., pentoses, such as, ribose, ribulose, arabinose, xylose, lyxose, and xylulose; hexoses, such as glucose, fructose, galactose, mannose, and sorbose; trioses; tetroses; heptoses; and octoses), disaccharide (e.g., sucrose, maltose, lactose, and trehalose), oligosaccharide, or a mixture thereof with one or more C₄-C₂₂ fatty acids (e.g., saturated fatty acids, such as caprylic acid, capric acid, lauric acid, myristic acid, palmitic acid, and stearic acid; and unsaturated fatty acids, such as palmitoleic acid, oleic acid, elaidic acid, erucic acid, and linoleic acid), and steroidal esters;

(iv) Ethers, for example, alkyl, aryl, and cyclic ethers having from 2 to about 30 carbons. Examples include diethyl ether, tetrahydrofuran, dimethyl isosorbide, diethylene glycol monoethyl ether), and glycofurol (tetrahydrofurfuranyl alcohol polyethylene glycol ether);

(v) Ketones which typically have from about 3 to about 30 carbons. Examples include acetone, methyl ethyl ketone, and methyl isobutyl ketone;

(vi) Hydrocarbons which are typically aliphatic, cycloaliphatic, or aromatic hydrocarbons having from about 4 to about 30 carbons. Examples include benzene, cyclohexane, dichloromethane, dioxolanes, hexane, n-decane, n-dodecane, n-hexane, sulfolane, tetramethylenesulfone, tetramethylenesulfoxide, toluene, dimethylsulfoxide (DMSO); and tetramethylene sulfoxide;

(vii) Oils which include, for example, oils of mineral, vegetable, animal, essential, or synthetic origin. These include: mineral oils, such as aliphatic and wax-based hydrocarbons, aromatic hydrocarbons, mixed aliphatic and aromatic based hydrocarbons, and refined paraffin oil; vegetable oils, such as linseed, tung, safflower, soybean, castor, cottonseed, groundnut, rapeseed, coconut, palm, olive, corn, corn germ, sesame, persic, and peanut oil; glycerides, such as mono-, di-, and triglycerides; animal oils, such as fish, marine, sperm, cod-liver, haliver, squaiene, squalane, and shark liver oil; oleic oils; and polyoxyethylated castor oil;

(viii) Alkyl, alkenyl, or aryl halides which include, for example, alkyl or aryl halides having from 1 to about 30 carbons and one or more halogen substituents. Examples include: methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (SOLUTOL HS-15, from BASF, Ludwigshafen, Germany); polyoxyethylene glycerol; sodium laurate; sodium oleate; and sorbitan monooleate.

Other pharmaceutically acceptable solvents for use in the invention are well known to those of ordinary skill in the art. General discussion relating to such solvents can be found in, for example, The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics 3d ed., (G. Banker et. al., eds., Marcel Dekker, Inc., New York, N.Y. (1995)), The Pharmacological Basis of Therapeutics, (Goodman & Gilman, McGraw Hill Publishing), Pharmaceutical Dosage Forms, (H. Lieberman et. al., eds., Marcel Dekker, Inc., New York, N.Y. (1980)), Remington's Pharmaceutical Sciences, 19th ed., (A. Gennaro, ed., Mack Publishing, Easton, Pa., (1995)), The United States Pharmacopeia 24, The National Formulary 19, (National Publishing, Philadelphia, Pa. (2000)); Spiegel, A. J., et al., “Use of Nonaqueous Solvents in Parenteral Products,” J. Pharma. Sciences, Vol. 52, No. 10, pp. 917-927 (1963).

Solvents useful in the invention include, but are not limited to, those known to stabilize present compounds or pharmaceutically acceptable salts thereof. These can include, for example, oils rich in triglycerides, such as safflower oil, soybean oil, and mixtures thereof; and alkyleneoxy-modified fatty acid esters, such as polyoxyl 40 hydrogenated castor oil and polyoxyethylated castor oils (e.g., CREMOPHOR EL solution or CREMOPHOR RH 40 solution). Commercially available triglycerides include INTRALIPID emulsified soybean oil (Kabi-Pharmacia Inc., Stockholm, Sweden), NUTRALIPID emulsion (McGaw, Irvine, Calif.), LIPOSYN II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), LIPOSYN III 2% emulsion (a 2% fat emulsion solution containing 100 mg safflower oil, 100 mg soybean oil, 12 mg egg phosphatides, and 25 mg glycerin per ml of solution; Abbott Laboratories, Chicago, Ill.), natural or synthetic glycerol derivatives containing the docosahexaenoyl group at levels of from about 25 to about 100% (by weight based on the total fatty acid content) (DHASCO from Martek Biosciences Corp., Columbia, Md.; DHA MAGURO from Daito Enterprises, Los Angeles, Calif.; SOYACAL; and TRAVEMULSION). Ethanol in particular is a useful solvent for dissolving a compound or pharmaceutically acceptable salt thereof to form solutions, emulsions, and the like.

Additional components can be included in the compositions of this invention for various purposes generally known in the pharmaceutical industry. These components tend to impart properties that, for example, enhance retention of the present compounds or salt thereof at the site of administration, protect the stability of the composition, control the pH, and facilitate processing of the compound or salt thereof into pharmaceutical formulations, and the like. Specific examples of such components include cryoprotective agents; agents for preventing reprecipitation of the compound or salt surface; active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stealyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide, etc.); and antioxidants (e.g., sodium metabisulfite, sodium bisulfite, sodium sulfite, dextrose, phenols, thiophenols, etc.).

Techniques and compositions for making parenteral dosage forms are generally known in the art. Formulations for parenteral administration can be prepared from one or more sterile powders and/or granules having a compound or salt of this invention and one or more of the carriers or diluents mentioned for use in the formulations for oral administration. The powder or granule typically is added to an appropriate volume of a solvent (typically while agitating (e.g., stirring) the solvent) that is capable of dissolving the powder or granule. Particular solvents useful in the invention include, for example, water, polyethylene glycol, propylene glycol, ethanol, corn oil, cottonseed oil, peanut oil, sesame oil, benzyl alcohol, sodium chloride, and/or various buffers.

Emulsions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the emulsion. Solutions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the solution.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, cocoa butter; synthetic mono-, di-, or triglycerides; fatty acids; and/or polyethylene glycols.

Every formulation or combination of components described or exemplified herein can be used to practice the invention, unless otherwise stated.

(i) Binding Agents:

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation.

(ii) Fillers:

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

(iii) Lubricants:

Lubricants include, but are not limited to, calcium stearate, magnesium stearate, mineral oil, electromagnetic radiation mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

(iv) Disintegrants:

Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Tablets or capsules can optionally be coated by methods well known in the art. If binders and/or fillers are used with a compound/bioconjugate of the invention, they are typically formulated as about 50 to about 99 weight percent of the compound/bioconjugate. In one aspect, about 0.5 to about 15 weight percent of disintegrant, and particularly about 1 to about 5 weight percent of disintegrant, can be used in combination with the compound. A lubricant can optionally be added, typically in an amount of less than about 1 weight percent of the compound/bioconjugate. Techniques and pharmaceutically acceptable additives for making solid oral dosage forms are described in Marshall, SOLID ORAL DOSAGE FORMS, Modern Pharmaceutics (Banker and Rhodes, Eds.), 7:359-427 (1979). Other formulations are known in the art.

Liquid preparations for oral administration can take the form of solutions, syrups or suspensions. Alternatively, the liquid preparations can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations can also contain buffer salts, flavoring, coloring, perfuming and sweetening agents as appropriate. Preparations for oral administration can also be formulated to achieve controlled release of the compound/bioconjugate. Oral formulations preferably contain 10% to 95% compound/bioconjugate. In addition, a compound/bioconjugate of the present invention can be formulated for buccal administration in the form of tablets or lozenges formulated in a conventional manner. Other methods of oral delivery of compounds/bioconjugates of the invention will be known to the skilled artisan and are within the scope of the invention.

Formulation 1

Hard gelatin capsules are prepared using the ingredients of Table F1. The ingredients of Table F1 are mixed and filled into hard gelatin capsules in 560 mg quantities.

Formulation 2:

A tablet formula is prepared using the ingredients of Table F2. The components are blended and compressed to form tablets, each weighing 665 mg.

Formulation 3:

A dry powder inhaler formulation is prepared containing the components of Table F3. The active ingredient is mixed with the lactose and the mixture is added to a dry powder inhaling appliance.

Formulation 4:

Tablets, each containing 60 mg of active ingredient, are prepared as outlined in Table F4. The active ingredient, starch and cellulose are passed through a No. 20 mesh U.S. sieve and mixed thoroughly. The solution of polyvinylpyrrolidone is mixed with the resultant powders which are then passed through a 16 mesh U.S. sieve. The granules as produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

Formulation 5:

Capsules, each containing 80 mg of active ingredient are made as indicated in Table F5. The active ingredient, cellulose, starch, and magnesium stearate are blended, passed through a No. 20 mesh U.S. sieve, and filled into hard gelatin capsules in 190 mg quantities.

Formulation 6:

Suppositories, each containing 225 mg of active ingredient, are made as shown in Table F6. The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Formulation 7

Suspensions, each containing 50 mg of active ingredient per 5.0 ml dose are made as shown in Table F7. The active ingredient, sucrose and xantham gum are blended, passed through a No. 10 mesh U.S. sieve, and mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Formulation 8:

Capsules, each containing 150 mg of active ingredient, are made as shown in Table F8. The active ingredient, cellulose, starch, and magnesium stearate are blended, passed through a No. 20 mesh U.S. sieve, and filled into hard gelatin capsules in 560 mg quantities.

4.e: Kits: Various embodiments of the present invention include kits. Such kits can include a compound/bioconjugate of the present invention, optionally one or more ingredients for preparing a pharmaceutically acceptable formulation of the compound/bioconjugate, and instructions for use (e.g., administration). When supplied as a kit, different components of a compound/bioconjugate formulation can be packaged in separate containers and admixed immediately before use. Such packaging of the components separately can, if desired, be presented in a pack or dispenser device which can contain one or more unit dosage forms containing the compound/bioconjugate. The pack can, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one combination for administration together.

It is further contemplated that the compounds and salts of this invention can be used in the form of a kit that is suitable for use in performing the methods described herein, packaged in a container. The kit can contain the compound or compounds and, optionally, appropriate diluents, devices or device components suitable for administration and instructions for use in accordance with the methods of the invention. The devices can include parenteral injection devices, such as syringes or transdermal patch or the like. Device components can include cartridges for use in injection devices and the like. In one aspect, the kit includes a first dosage form including a compound or salt of this invention and a second dosage form including another active ingredient in quantities sufficient to carry out the methods of the invention. The first dosage form and the second dosage form together can include a therapeutically effective amount of the compounds for treating the targeted condition(s).

In certain embodiments, kits can be supplied with instructional materials. Instructions can be printed on paper or other substrate, and/or can be supplied as an electronic-readable medium, such as a floppy disc, mini-CD-ROM, CD-ROM, DVD-ROM, Zip disc, videotape, audio tape, and the like. Detailed instructions cannot be physically associated with the kit; instead, a user can be directed to an Internet web site specified by the manufacturer or distributor of the kit, or supplied as electronic mail.

If desired, the emulsions or solutions described above for oral or parenteral administration can be packaged in IV bags, vials, or other conventional containers in concentrated form, and then diluted with a pharmaceutically acceptable liquid (e.g., saline) to form an acceptable compound concentration before use.

Kits can include reagents in separate containers such as, for example, sterile water or saline to be added to a lyophilized active component packaged separately. For example, sealed glass ampules can contain lyophilized superoxide dismutase mimetics and in a separate ampule, sterile water, sterile saline or sterile each of which has been packaged under a neutral non-reacting gas, such as nitrogen. Ampules can consist of any suitable material, such as glass, organic polymers, such as polycarbonate, polystyrene, ceramic, metal or any other material typically employed to hold reagents. Other examples of suitable containers include bottles that can be fabricated from similar substances as ampules, and envelopes that can consist of foil-lined interiors, such as aluminum or an alloy. Other containers include test tubes, vials, flasks, bottles, syringes, and the like. Containers can have a sterile access port, such as a bottle having a stopper that can be pierced by a hypodermic injection needle. Other containers can have two compartments that are separated by a readily removable membrane that upon removal permits the components to mix. Removable membranes can be glass, plastic, rubber, and the like.

### Example: Treatment of Acne Vulgaris

Any of the methods of treating provided herein may be used to treat acne vulgaris. This is example is based in part on a recognition that acne vulgaris appears associated with Demodex mites. See, e.g., Zhao et al. J. Zhejiang Univ-Sci B (Biomed & Biotechnol. 2012 13(3):192-202. Accordingly, any of the compounds and compositions described herein that kill demodex mites may be used in a method of treating acne vulgaris.

### Example

Diagnostic tests for skin affliction caused by Demodex mites. Interestingly, initial treatment applications with a chloroquine and/or hydroxychloroquine results in an observable flare-up response in certain applied areas. After initial treatment, not observable flare-up occurs. Similarly, patients that do not have the skin affliction do not have an observable flare-up response after chloroquine and/or hydroxychloroquine application. A reason for these results is that patients that do not have a Demodex mite infestation, or that are not sensitized to a bacteria carried by the Demodex mites, do not have an initial adverse response to chloroquine and/or hydroxychloroquine treatment. This difference in response to chloroquine and/or hydroxychloroquine may be employed as a tool to diagnose a skin affliction. For example, a skin affliction that is rosacea may be diagnosed by initial short-term application of a chloroquine and/or hydroxychloroquine. If there is an observable flare-up response, such as substantial flushing, redness, bloating, discoloration, itching or other irritation, a diagnosis of a mite-related skin affliction, such as rosacea, may be made. Alternatively, if there is no observable flare-up response, a negative diagnosis may be made. The application can be by any reliable means to the skin, similar to a skin allergy test. The chloroquine and/or hydroxychloroquine may be applied to a surface of an application substrate having an adhesive side that supports the active agent that is applied to the skin for an application time, such as an application time that is greater than 10 seconds and less than about 5 minutes. The application substrate is removed and the skin observed for a flare-up response. A positive flare-up response indicates the presence of a skin affliction, including presence of mites that are susceptible to the chloroquine and/or hydroxychloroquine. In contrast, if no flare-up response is observed, the patient is characterized as not having the skin affliction and/or not having the mites that are susceptible to the chloroquine and/or hydroxychloroquine. Other types of application steps are compatible in the diagnostic methods of the instant invention, including swabbing, or self-application. The diagnostic test may further comprise the step of rinsing the skin to remove the chloroquine and/or hydroxychloroquine prior to the observation step. Controls may be used, with an identical application except without the chloroquine and/or hydroxychloroquine to control for other allergic responses associated with the diagnostic test (e.g., latex and/or adhesive sensitivity). The application area may be selected to be a localized area, such as less than about 100 cm², less than 10 cm², or less than 1 cm². Preferably, the area corresponds to regions where the mites tend to favor, including certain facial areas.

For patients having an observable flare-up response, the patient may then be treated with a full course of chloroquine and/or hydroxychloroquine treatment. For patients not having an observable flare-up response, a treatment that is not a chloroquine and/or hydroxychloroquine treatment may be indicated.

The chloroquine and/or hydroxychloroquine may correspond to a compound currently used in medicine, including those having an established safety profile in humans, including PLAQUENIL® hydroxychloroquine.

### Example

In vitro efficacy and mite lifetimes: Relative efficacy of various formulations are evaluated with a demodex survival time experiments. FIG. 3 tabulates demodex survival time, expressed in terms of LT50 (time at which 50% of demodex mites are killed) and/or average minutes to death for various active agents. The compounds having an LT50 labeled “no activity” reflects an activity that is not significantly different from control (e.g., water).

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, and method steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim. PCT Pub. Nos. WO 2015/017328 and 2015/195928 are specifically incorporated by reference herein, including for formulations, methods, therapeutics and administration.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to or “‘2’ or ‘3’ or ‘1 and 2’ or ‘1 and 3’ or ‘2 and 3’ or ‘1, 2 and 3’”.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range. As used herein, ranges specifically include all the integer values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed can be resorted to by those skilled in the art, and that such modifications and variations are considered to be within the scope of this invention as defined by the appended claims.

Every formulation or combination of components described or exemplified can be used to practice the invention, unless otherwise stated. Specific names of materials are intended to be exemplary, as it is known that one of ordinary skill in the art can name the same material differently. One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

### Tables

TABLE F1

| Ingredients | (mg/capsule) |
|---|---|
| Active Ingredient | 250.0 |
| Starch | 305.0 |
| Magnesium stearate | 5.0 |

TABLE F2

| Ingredients | (mg/tablet) |
|---|---|
| Active Ingredient | 250.0 |
| Cellulose, microcrystalline | 400.0 |
| Colloidal silicon dioxide | 10.0 |
| Stearic acid | 5.0 |

TABLE F3

| Ingredients | Weight % |
|---|---|
| Active ingredient | 5 |
| Lactose | 95 |

TABLE F4

| Ingredients | Milligrams |
|---|---|
| Active ingredient | 60.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |
| Total | 150.0 |

TABLE F5

| Ingredients | Milligrams |
|---|---|
| Active ingredient | 80.0 |
| Starch | 109.0 |
| Magnesium stearate | 1.0 |
| Total | 190.0 |

TABLE F6

| Ingredients | Milligrams |
|---|---|
| Active Ingredient | 225 |
| Saturated fatty acid glycerides to | 2000 |

TABLE F7

| Ingredients | Milligrams | |
|---|---|---|
| Active ingredient | 50.0 | mg |
| Xanthan gum | 4.0 | mg |
| Sodium carboxymethyl cellulose | (11%) | |
| Microcrystalline cellulose | (89%) 50.0 mg | |
| Sucrose | 1.75 | g |
| Sodium benzoate | 10.0 | mg |
| Flavor | q.v. | |
| Color | q.v. | |
| Purified water to | 5.0 | ml |

TABLE F8

| Ingredients | Milligrams |
|---|---|
| Active ingredient | 150.0 |
| Starch | 407.0 |
| Magnesium stearate | 3.0 |
| Total | 560.0 |

TABLE OF REFERENCES
[1] Nat'l Rosacea Soc.: www.rosacea.org/patients/allaboutrosacea.php
[2] Kligman A M & Christensen M S. (2011) Demodex folliculorum:
Requirements for Understanding Its Role in Human Skin Disease. Journal of
Investigative Dermatology. 131: 8-10
[3] Despommier, D, Gwadz R W, Hotez P J and Knirsch C A. Parasitic
Diseases. 5th ed. New York: Apple Trees Production, LLC. 2006
[4] Hsu C K, Hsu M M, Lee J Y. (2009) Demodicosis: a
clinicopathological study. J Am Acad Dermatol. 60(3): 453-62
[5] Lacey N, Kavanagh K, Tseng S C. (2009) Under the lash: Demodex
mites in human diseases. Biochem (Lond). 31(4): 2-6
[6] Liua J, Sheha H, & Tsenga S C G. (2010) Pathogenic role of
Demodex mites in blepharitis. Curr Opin Allergy Clin Immunol. 10(5): 505-510.
[7] Thomson, W. T. 1976. Agricultural chemicals - book 1:
insecticides, acaricides, and ovicides. Revised ed. Thomson Publ., Indianapolis, IN.
232 pp.
[8] The Pesticide Manual: A World Compendium, 7th ed. 1983. C.R.
Worthing, ed. The British Crop Protection Council, Croydon, England. 695 pp.
[9] U.S. Department of Health and Human Services, National Institute
for Occuptational Safety and Health. 1981. Occupational health guidelines for
chemical hazards. F. W. Mackinson, R. S. Stricoff, L. J. Partridge, Jr., and A. D.
Little, Inc., eds. DHHS (NIOSH) Publ. No. 81-123. Washington, DC.
[10], American Conference of Governmental Industrial Hygienists.
1984. TLVs: threshold limit values for chemical substances and physical agents in
the work environment and biological exposure indices with intended changes for
1984-85. Cincinnati, OH. 116 pp.
[11] Farm Chemicals Handbook, 70th ed. 1984. R. T. Meister, G. L.
Berg, C. Sine, S. Meister, and J. Poplyk, eds. Meister Publishing Co., Willoughby,
OH.
[12] Demodex mites: Facts and controversies. Elston D M.
Department of Dermatology, Geisinger Medical Center, 100 N
Academy Ave, Danville, Danville, PA 17822-5206, USA. Clin Dermatol. 2010
September-October; 28(5): 502-504.
[13] For a partial list of research articles on demodectic rosacea
www.irosacea.org/html/demodex_rosacea_research_papers.html
[14] Beating Rosacea Vascular, Ocular Acne Forms, page 110
Geoffrey Nase, Ph.D. Nase Publications 2001
[15] Mar. 28, 2007 R-S post by David Pascoe
[16] Rosacea Review, Fall 2010, NRS-Funded Studies Advance
Knowledge of Rosacea's Causes
[17] Rosaceam Like Demodicidosis, SAMUEL AYRES, JR., M.D., Los
AngelesCalifornia Medicine, June 1963
[18] Something to Blush About, Medical Breakthoughs, Ivanhoe
Newswire, Dec. 11, 2007
[19] Rosacea Diagnosis and Management, pages 69, 70 by Frank C.
Powell, Informa Healthcare, 2008
[20] Empirical treatment is key to identifying rosacea, other
dermatoses, Modern Medician, John Jesitus, Nov. 1, 2007
[21] Demodicosis and rosacea: epidemiology and significance in daily
dermatologic practice. Forton F, Germaux M A, Brasseur T, De Liever A, Laporte M,
Mathys C, Sass U, Stene J J, Thibaut S, Tytgat M, Seys B. J Am Acad Dermatol.
2005 January; 52(1): 74-87.
[22] A clinico-pathological approach to the classification of human
demodicosis. Akilov O E, Butov Y S, Mumcuoglu K Y. J Dtsch Dermatol Ges. 2005
August; 3(8): 607-14.
[23] Could matrix metalloproteinase-9 be a link between Demodex
folliculorum and rosacea? R R Bonamigo, L Bakosl, M Edelweiss, A Cartell Journal
of the European Academy of Dermatology & Venereology, Volume 19 Issue 5 Page
646 - September 2005
[24] Is Demodex really non-pathogenic? Pena G P, Andrade Filho J S
Rev Inst Med Trop Sao Paulo. 2000 May-June; 42(3): 171-3.
[25] Facial demodicosis. Zomorodian K, Geramishoar M, Saadat F,
Tarazoie B, Norouzi M, Rezaie S. Eur J Dermatol. 2004 March-April; 14(2): 121-2.
[26] Demodicidosis revisited. Baima B, Sticherling M. Acta Derm
Venereol. 2002; 82(1): 3-6
[27] Rosacea, eMedicine from WebMD Author: Agnieszka Kupiec
Banasikowska, MD, Consulting Staff, Georgetown Dermatology, PLLC Coauthor(s):
Saurabh Singh, MD, Staff Physician, Department of Dermatology, Georgetown
University/Washington Hospital Center
[28] Rosacea and the pilosebaceous follicle. Powell F C. Cutis. 2004
September; 74(3 Suppl): 9-12, 32-4.
[29] Mite-related bacterial antigens stimulate inflammatory cells in
rosacea. Lacey N, Delaney S, Kavanagh K, Powell F C. Br J Dermatol. 2007
September; 157(3): 474-81. Epub 2007 Jun. 26
[30] New Study Shows Role for Bacteria in Development of Rosacea
Symptoms National Rosacea Society, May 3, 2004
[31] Frequency of the appearance of Demodex sp. in various patient
and age groups Aycan OM, Otlu G H, Karaman U, Daldal N, Atambay M. Turkiye
Parazitol Derg. 2007; 31(2): 115-8.
[32] Electronmicroscopic investigation into the possible etiology of
rosacea and the implication for treatment Journal of the American Academy of
Dermatology February 2007 (Vol. 56, Issue 2 (Supplement 2), Page AB44) Richard
Burroughs, MD, National Capital Consortium (Walter Reed Army Medical Center),
Washington, DC, United States; Kurt Maggio, MD, Walter Reed Army Medical
Center, Washington, DC, United States
[33] A study on Demodex folliculorum in rosacea. Abd-El-Al A M,
Bayoumy A M, Abou Salem E A. J Egypt Soc Parasitol. 1997 April; 27(1): 183-95.
[34] The pathogenesis of Demodex folliculorum (hair follicular mites) in
females with and without rosacea. el-Shazly A M, Ghaneum B M, Morsy T A, Aaty H E.
J Egypt Soc Parasitol. 2001 December; 31 (3): 867-75.
[35] Demodex mites in acne rosacea. Roihu T, Kariniemi A L. J Cutan
Pathol. 1998 November; 25(10): 550-2.
[36] The possible role of skin surface lipid in rosacea with epitheloid
granulomas. Basta-Juzbasi&#263; A, Marinovi&#263; T, Dobri&#263; I, Bolanca-
Bumber S, Sencar J.Acta Med Croatica. 1992; 46(2): 119-23.
[37] Study finds cause of rosacea Claire O'Connell The Irish Times -
Tuesday, Jul. 14, 2009
[38] New Study Shows Role for Bacteria in Development of Rosacea
Symptoms NRS Press Release, May 3, 2004, Suzanne Corr/Barbara Palombo
[39] Mite-related bacterial antigens stimulate inflammatory cells in
rosacea. Lacey N, Delaney S, Kavanagh K, Powell F C. Department of Biology,
National University of Ireland, Maynooth, Co. Kildare, Ireland Br J Dermatol. 2007
September; 157(3): 474-81
[40] Correlation between Ocular Demodex Infestation and Serum
Immunoreactivity to Bacillus Proteins in Patients with Facial Rosacea, Li J, O'Reilly
N, Sheha H, Katz R, Raju V K, Kavanagh K, Tseng S C. Ophthalmology. 2010 Jan. 14,
Papulopustular rosacea, skin immunity and Demodex: pityriasis folliculorum as a
missing link. Forton F M. J Eur Acad Dermatol Venereol. 2011 Oct. 24. doi:
10.1111/j.1468-3083.2011.04310.x. &copy; 2011 The Author. Journal of the
European Academy of Dermatology and Venereology &copy; 2011 European
Academy of Dermatology and Venereology. PMID: 22017468 [PubMed - as supplied
by publisher]
[42] Demodex Infestation: the Missing Link ? Oct. 27th, 2011, by
David Pascoe
[43] Demodex Mites, Ivermectin (Stromectol) and its use in
Dermatology
Feb. 2nd, 2006, by David Pascoe
[44] Rosacea: Diagnosis and Management Frank Powell, MD, Informa
Healthcare, 2008
[45] Intense Pulsed Light Eradicates Demodex Mites Timothy F. Kirn
Sacramento Bureau, Sharon Worcester, Tallahassee bureau, contributed this story
&bull; Another source
[46] Rejecting common wisdom: Red, scaly faces not always rosacea
or seborrheic dermatitis Dermatology Times, Modern Medicine, Jane Schwanke,
Jun. 1, 2009
[47] Treatment of human Demodex folliculorum by camphor oil and
metronidazole. El-Shazly A M, Hassan A A, Soliman M, Morsy G H, Morsy T A. J Egypt
Soc Parasitol. 2004 April; 34(1): 107-16.
[48] Eucalyptus globulus (camphor oil) in the treatment of human
demodicidosis. Morsy T A, Morsy G H, Sanad E M. J Egypt Soc Parasitol. 2002
December; 32(3): 797-803
[49] Read the report in footnote [10] above
[50] Ivermectin: pharmacology and application in dermatology
International Journal of Dermatology, Volume 44 Page 981 - December 2005.
Pharmacology and therapeutics; Assen L. Dourmishev, Lyubomir A. Dourmishev,
and Robert A. Schwartz
[51] Natural remedies aid rosacea management, doctor says Dietary
changes, supplements can lead to clearer skin Lisette Hilton, Dermatology Times,
Modern Medicine, Dec. 1, 2004
[52] In vitro and in vivo killing of ocular Demodex by tea tree oil. Gao
Y Y, Di Pascuale M A, Li W, Baradaran-Rafii A, Elizondo A, Kuo C L, Raju V K, Tseng
S C Br J Ophthalmol. 2005 November; 89(11 ): 1468-73.
[53] Lavender and Tea Tree Oils May Cause Breast Growth in Boys
NIH News, Wednesday, Jan. 31, 2007
[54] Non-invasive in vivo detection and quantification of Demodex
mites by confocal laser scanning microscopy. Sattler E C, Maier T, Hoffmann V S,
Hegyi J, Ruzicka T, Berking C. Br J Dermatol. 2012 Jun. 20. doi: 10.1111/j.1365-
2133.2012.11096.x.
[55] Counting Demodex Mites with a Confocal Laser Microscope David
Pascoe Rosacea Support Group
[56] Wistar's comment on Demodex testing post #4 Apr. 10, 2011 at
10:01 PM
[57] Unilateral demodectic rosacea. Shelley W B, Shelley E D,
Burmeister V. J Am Acad Dermatol. 1989 May; 20(5 Pt 2): 915-7.
[58] Demodex Dermatitis A Retrospective Analysis of Clinical
Diagnosis and Successful Treatment with Topical Crotamiton Joseph B. Bikowski,
MD, FAAD and James Q. Del Rosso, DO, FAOCD Journal List >J Clin Aesthet
Dermatol >v.2(1); January 2009 >PMC2958185
[59] The Chicken, not the Egg? National Rosacea Society, Thursday,
Jul. 5, 2012
[60] More Demodex Dreaming: Mites are the Chicken? by David
Pascoe, July 24th, 2012
[61] Br J Dermatol. 2013 Feb. 16. doi: 10.1111/bjd.12280. Demodex
quantification methods: Limitations of Confocal Laser Scanning Microscopy (CLSM).
Lacey N, Forton F M, Powell F C.
[62] Indian J Dermatol. 2013 March; 58(2): 157. doi: 10.4103/0019-
5154.108069. Evaluation of Demodex folliculorum as a Risk Factor for the Diagnosis
of Rosacea In Skin Biopsies. Mexico's General Hospital (1975-2010). Rios-Yuil
J M, Mercadillo-Perez P.
[63] The Quality of Life Impact of Acne and Rosacea Compared to
Other Major Medical Conditions. Nicole D. Cresce BS, Scott A. Davis MA, William
W. Huang MD MPH, Steven R. Feldman MD PhD. Journal of Drugs and
Dermatology June 2014.
[64] An Evaluation of the Potential Correlations Between
Pathophysiologic Mechanisms, Clinical Manifestaions, and Managemant of
Rosacea. James Q. Del Rosso, DO; Richard L Gallo, MD, PhD; Emil Tanghetti,
MD; Guy Webster, MD, PhD; Diane Thiboutot, MD. Cutanous Medicine For The
Practicioner. vol. 91 no. 3s March 2013.
[65] Influence of temperature and medium on viability of Demodex
folliculorum and Demodex brevis. Ya-E Zhao; Na Guo; Li-Ping Wu; Exp Appl Acarol
(2011) 54: 421-425
[66] The effect of temperature on the viability of Demodex folliculorum
and Demodex brevis. Ya-E Zhao; Na Guo; Li-Ping Wu; Parasitol Research (2009)
105: 1623-1628
www.youtube.com/watch?v=rl-L79szJ6Q&feature=related
www.youtube.com/watch?feature=endscreen&NR=1&v=pZof6KvD88g
www.youtube.com/watch?v=3LLk7CNIYdQ
www.psmicrographs.co.uk/follicle-mite--Demodex-folliculorum-/science-
image/80016342
Are Mites Causing Your Rosacea?: http://www.webmd.com/skin-
problems-and-treatments/news/20120830/are-mites-causing-your-rosacea
Mites May Cause Rosacea and Botox May Cure Acne in Alluring Links:
http://www.allure.com/beauty-trends/blogs/daily-beauty-reporter/2012/08/beauty-
news-week-of-august-31.html
Rosacea May Be Caused By Bacteria Released By Tiny Mites Living
On The Skin: http://www.medicalnewstoday.com/releases/249664.php
Could Bacteria in Skin Mites Help Cause Rosacea?:
http://health.usnews.com/health-news/news/articles/2012/08/30/could-bacteria-in-
skin-mites-help-cause-rosacea
Bacterial Cause Found for Skin Condition Rosacea:
http://members.rosacea-research-and-development-institute.org/topic/1297-
demodectic-rosacea-in-the-media/
Tiny mites on your face may cause rosacea:
http://vitals.nbcnews.com/_news/2012/08/29/13554038-tiny-mites-on-your-face-may-
cause-rosacea?lite
Rosacea: Caused by Mite Poop in Your Facial Pores?:
http://healthland.time.com/2012/09/04/rosacea-caused-by-mite-poop-in-your-facial-
pores/
Rosacea may be caused by mite faeces in your pores:
http://www.newscientist.com/article/dn22227-rosacea-may-be-caused-by-mite-
faeces-in-your-pores.html
Bacteria-laden mites may cause rosacea:
http://dermatologytimes.modernmedicine.com/dermatologytimes/Modem+Medicine+
News/Bacteria-laden-mites-may-cause-
rosacea/ArticleStandard/Article/detail/787502?ref=25
New discovery may hold clues to rosacea cure; Red bumps may be
linked to mites living on the face: http://www.nydailynews.com/life-style/health/new-
discovery-hold-clues-rosacea-cure-red-bumps-linked-mites-living-face-article-
1.1152511#ixzz25klcWNDW
Rosacea caused by bacteria from tiny mites?:
http://abclocal.go.com/wls/story?section=news/health&id=8792589
Health & Beauty: Could These Tiny Mites Be Causing Your Rosacea?:
http://www.glamour.com/health-fitness/blogs/vitamin-g/2012/08/health-beauty-could-
these-tiny.html#ixzz25kK66pNH
Rosacea Comes From Mite Feces:
http://www.theatlanticwire.com/technology/2012/08/strange-heartland-virus-
discovered-rosacea-comes-mite-feces/56390/
That Rose in Your Cheeks Could Be Bacteria:
http://abcnews.go.com/blogs/health/2012/08/29/that-rose-in-your-cheeks-could-be-
bacteria/
Rosacea may be caused by skin bacteria: study: http://www.business-
standard.com/generalnews/news/rosacea-may-be-caused-by-skin-bacteria-
study/49985/
Researchers Claim to be Closer Towards Effective Treatement of
Rosacea: http://www.business-standard.com/generalnews/news/rosacea-may-be-
caused-by-skin-bacteria-study/49985/
Face Bacteria Only Cause Deadly Skin Disease:
http://frenchtribune.com/teneur/1213176-face-bacteria-only-cause-deadly-skin-
disease
The secret to rosacea: mite poop in you pores:
http://www.smartplanet.com/blog/rethinking-healthcare/the-secret-to-rosacea-mite-
poop-in-you-pores/9936
Rosacea caused by mite poop:
http://www.theatlanticwire.com/technology/2012/08/strange-heartland-virus-
discovered-rosacea-comes-mite-feces/56390/
Could Bacteria in Skin Mites Help Cause Rosacea?:
http://www.newsday.com/news/health/could-bacteria-in-skin-mites-help-cause-
rosacea-1.3938011
Rosacea may be caused by mite faeces in your pores:
www.newscientist.com/article/dn22227-rosacea-may-be-caused-by-mite-faeces-in-
your-pores.html
Red skin condition rosacea may be due to bacteria in skin mites:
www.cbsnews.com/8301-504763_162-57503771-10391704/red-skin-condition-
rosacea-may-be-due-to-bacteria-in-skin-mites/
Rosacea: A product of bacteria from mites?:
http://www.foxnews.com/health/2013/02/05/rosacea-product-bacteria-from-mites/
Scientists Find Cause Of Rosacea, And It's Terrifying:
http://www.buzzfeed.com/jtes/scientists-find-cause-of-rosacea-and-its-ter
Indian J Dermatol. 2013 March; 58(2): 157. doi: 10.4103/0019-
5154.108069. Evaluation of Demodex folliculorum as a Risk Factor for the Diagnosis
of Rosacea In Skin Biopsies. Mexico's General Hospital (1975-2010). Rios-Yuil J M,
Mercadillo-Perez P.x.
Positive correlation between serum immuno-reactivity to Demodex-
associated Bacillus proteins and Erythematotelangiectic Rosacea. O'Reilly N,
Menezes N, Kavanagh K. Br J Dermatol. 2012 Jun. 18. doi: 10.1111/j.1365-
2133.2012.11114.x.
Quantification of Demodex folliculorum by PCR in rosacea and its
relationship to skin innate immune activation. Roman-Curto C, Meseguer-Yebra C,
Canueto J, Fraile-Alonso C, Santos-Briz A, Vazquez L, Fernandez-Lopez E. Transpl
Infect Dis. 2012 Apr. 9. doi: 10.1111/j.1399-3062.2012.00729.x
Demodicidosis simulating acute graft-versus-host disease after
allogeneic stem cell transplantation in one patient with acute lymphoblastic leukemia.
Casas C, Paul C, Lahfa M, Livideanu B, Lejeune O, Alvarez-Georges S, Saint-
Martory C, Degouy A, Mengeaud V, Ginisty H, Durbise E, Schmitt A M, Redoulès D.
Exp Dermatol. 2012 December; 21(12): 906-10. doi: 10.1111/exd.12030
Risk factors and prevalence of Demodex mites in young adults.
Horvath A, Neubrandt D M, Ghidan A, Nagy K Acta Microbiol Immunol Hung. 2011
June; 58(2): 145-55. Demodex-associated bacterial proteins induce neutrophil
activation. O'Reilly N, Bergin D, Reeves E P, McElvaney N G, Kavanagh K. Br J
Dermatol. 2011 Nov. 19. doi: 10.1111/j.1365-2133.2011.10746.x
Papulopustular rosacea, skin immunity and Demodex: pityriasis
folliculorum as a missing link. Forton F M. J Eur Acad Dermatol Venereol. 2011 Oct.
24. doi: 10.1111/j.14683083.2011,04310.x
Retrospective analysis of the association between Demodex infestation
and rosacea. Zhao Y E, Wu L P, Peng Y, Cheng H. Arch Dermatol. 2010
August; 146(8): 896-902.
Comparison of the two techniques for measurement of the density of
Demodex folliculorum: standardized skin surface biopsy and direct microscopic
examination. Askin U, Segkin D. Br J Dermatol. 2010 Feb. 25
Correlation between Ocular Demodex Infestation and Serum
Immunoreactivity to Bacillus Proteins in Patients with Facial Rosacea Li J, O'Reilly N,
Sheha H, Katz R, Raju V K, Kavanagh K, Tseng S C. Ophthalmology. 2010 Jan. 14.
Pathogenic role of Demodex mites in blepharitis Jingbo Liu, Hosam
Sheha, and Scheffer C. G. Tsegn Curr Opin Allergy Clin Immunol. 2010 October;
10(5): 505-510.
Demodicosis: a clinicopathological study. Hsu C K, Hsu M M, Lee J Y. J
Am Acad Dermatol. 2009 March; 60(3): 453-62.
Under the lash Demodex mites in human diseases Noreen Lacey,
Kevin Kavanagh, and Scheffer C. G. Tseng Biochem (Lond). 2009 Aug. 1; 31(4):
2-6.
Facial Demodex infection among college students in Tangshan Cao
Y S, You Q X, Wang L, Lan H B, Xu J, Zhang X H, Yang H, Xiong Y J, Tian X F.
Laboratory of Medical Examination, Biosciences Department of North China Coal
Medical University, Tangshan 063000, China. Zhongguo Ji Sheng Chong Xue Yu Ji
Sheng Chong Bing Za Zhi. 2009 June; 27(3): 271-3.
Rosacea-Like Demodicosis Induced by Topical Pimecrolimus:
Immunohistochemical Evaluation of Inflammatory Infiltrate Efi Pasmatzi, MD, Maria
Melachrinou, MD, Alexandra Monastirli1, MD, Anastasia Tzouma1, MD, Dionysios
Tsambaos1, MD, PhD HOSPITAL CHRONICLES 2009, 4(4): 172-174
Rosacea-like demodicidosis. Larios G, Alevizos A, Perimeni D,
Rigopoulos D, Katsambas A. Department of Dermatology, University of Athens
Medical School, Andreas Sygros Hospital, Athens, Greece. Lancet Infect Dis. 2008
December; 8(12): 804.
Clinical importance of Demodex folliculorum in patients receiving
phototherapy. Kulac M, Ciftci I H, Karaca S, Cetinkaya Z Int J Dermatol. 2008
January; 47(1): 72-7
Mite-related bacterial antigens stimulate inflammatory cells in rosacea.
Lacey N, Delaney S, Kavanagh K, Powell F C. Department of Biology, National
University of Ireland, Maynooth, Co. Kildare, Ireland. Br J Dermatol. 2007 Jun. 26
Rosacea. Author: Agnieszka Kupiec-Banasikowska, MD, Consulting
Staff, Division of Dermatology, Georgetown University Medical Center Coauthor(s):
Mana Ogholikhan, MD, Staff Physician, Division of Dermatology, Georgetown
University Hospital; Ravi Ratnavel, MD, Consulting Staff, Department of
Dermatology, Stoke Mandeville, Thames Valley Nuffield, Paddocks Hospitals, UK-
2007
Association of rosacea with demodicosis. Morawej H, Dehghan-
Mangabadi M, Abbasian M R, Meshkat-Razavi G: Arch Iran Med. 2007
April; 10(2): 199-203.
Corneal manifestations of ocular Demodex infestation Kheirkhah A,
Casas V, Li W, Raju V K, Tseng S C. Am J Ophthalmol. 2007 May; 143(5): 743-749.
Epub 2007 Mar. 21
Demoodex folliculorum and Demodex brevis as a cause of chronic
marginal blepharitis. Czepita D, Ku?na-Grygiel W, Czepita M, Grobelny A. Ann Acad
Med Stetin. 2007; 53(1): 63-7; discussion 67.
Granulomatous rosacea-like demodicidos Julia Yu-Yun Lee MD, Chao-
Kai Hsu MD Dermatology Online Journal 13 (4): 9; 2007, Vol 13, No. 1
Standardized skin surface biopsy: method to estimate the Demodex
folliculorum density, not to study the Demodex folliculorum prevalence. Forton F. J
EurAcad Dermatol Venereol. 2007 October; 21(9): 1301-2
Density of Demodex folliculorum in rosacea: a case-control study using
standardized skin-surface biopsy. F. FORTON 1 B. SEYS. Clinic of Dermatology,
Saint Pierre University Hospital, Université Libre de Bruxetles, Brussels, Belgium,
Unit of Medical Sciences Pedagogy, Faculty of Medicine, Université Catholique de
Louvain, Brussels, Belgium British Journal of Dermatology, Volume 128 Issue 6,
Pages 650-659. Published Online: 29 Jul. 2006
Demodicosis and rosacea: Epidemiology and significance in daily
dermatologic practice, Journal of the American Academy of Dermatology, Volume
52, Issue 1, January 2005, Pages 74-87, Forton F, Germaux M A, Brasseur T, De
Liever A, Laporte M, Mathys C, Sass U, Stene J J, Thibaut S, Tytgat M, Seys B.
Structural and biological changes in rosacea skin induced by the
595 nm long-pulse dye laser and intense pulsed light. Dr. Payam Tristani-Firouzi,
assistant professor, and Dr. Nancy Samolitis, visiting professor, department of
dermatology, University of Utah. This study being done in 2005 by a grant from the
NRS will study among other things and “assess the size of the oil glands and the
presence of Demodex mites, normal inhabitants of human skin that have been
observed in greater numbers in rosacea patients.”
Demodecidosis in a patient infected by HIV: successful treatment with
ivermectin. Clyti E, Sayavong K, Chanthavisouk K: Ann Dermatol Venereol. 2005
May; 132(5): 459-61. Service de Dermatologie, Institut Guyanais de Dermatologie
Tropicale, Hopital de Cayenne, Guyane Francaise.
Density of Demodex folliculorum in perioral dermatitis. Dolenc-Voljc M,
Pohar M, Lunder T: Acta Derm Venereol. 2005; 85(3): 211-5: Department of
Dermatovenereology, University Medical Centre Ljublana, Zaloska 2, SI-1525
Ljublana, Slovenia.
Demodicidosis in humans as a current problem in dermatology: Wiad
Parazytol. 2005; 51(3): 253-6.
In vitro and in vivo killing of ocular Demodex by tea tree oil. Gao Y Y, Di
Pascuale M A, Li W, Baradaran-Rafii A, Elizondo A, Kuo C L, Raju V K, Tseng S C: Br
J Ophthalmol. 2005 November; 89(11): 1468-73.
Demodex mites as a cause of human disease. Elston D M; Cutis. 2005
November; 76(5): 294-6.
Demodex as an etiological factor in chronic blepharitis Czepita D,
Ku?na-Grygiel W, Kosik-Bogacka D. Klin Oczna. 2005; 107(10-12): 722-4.
Biochemical and immunological characterization of the role of bacterial
antigens in the induction of papulopustular rosacea. Dr. Kevin Kavanagh,
Department of biology, National University of Ireland, Maynooth, and Dr. Frank
Powell, Consultant Dermatologist, Mater Misericordiae Hospital, Dublin. 2004. Dr.
Kevin Kavanagh was awarded $25,000 to pursue further research on the potential
role of bacterial antigens in papulopustular (subtype 2) rosacea. In an earlier NRS-
funded study, he and his colleagues succeeded in isolating a bacterium from
Demodex folliculorum, microscopic mites that are a common inhabitant of facial skin.
The bacteria produced antigens that induced an inflammatory response in
significantly more rosacea patients than controls. In the new study, they will
determine whether the presence of the antigens is predictive of the onset of rosacea,
in order to establish whether they play a significant role.
Relationship between the Demodex and bacteria infection in human
rosacea. Hu Q, Wang Y, Tong L: Zhongguo Ji Sheng Chong Xue Yu Ji Sheng
Chong Bing Za Zhi. 2004 Feb. 28; 22(1): 50-3. Department of Parasitology, Medical
college of Inner Mongolia National University, Tongliao 028041, China.
New Study Shows Role for Bacteria in Development of Rosacea
Symptoms. Suzanne Corr/Barbara Palombo; PROVIDENCE, R.I. (May 3, 2004):
National Rosacea Society
Studies of di-n-butyl phthalate-OP emulsion in the treatment of
demodicidosis. Xia H, Hu S F, Ma W J, Ge J H: Zhongguo Ji Sheng Chong Xue Yu Ji
Sheng Chong Bing Za Zhi. 2004 August; 22(4): 248-9. Department of Microbiology and
Parasitology, Bengbu Medical College, Bengbu 233003, China.
Rosacea: a clinicopathological approach. Aroni K, Tsagroni E, Lazaris
A C, Patsouris E, Agapitos E; Dermatology. 2004; 209(3): 177-82. Department of
Dermatopathology, School of Medicine, National and Kapodistrian University of
Athens
Immune response in demodicosis. Akilov O E, Mumcuoglu K Y; J Eur
Acad Dermatol Venereol. 2004 July; 18(4): 440-4. Department of Dermatology,
Cosmetology Hospital ‘Aesthetics’, Ekaterinburg, Russian Federation.
Demodex abscesses: clinical and therapeutic challenges. Schaller M,
Sander C A, Plewig G: J Am Acad Dermatol. 2003 November; 49(5 Suppl): S272-4.
Department of Dermatology and Allergology, University of Munich, Germany
Some aspects of the skin infestation by Demodex folliculorum n; Wiad
Parazytol. 2004; 50(1): 41-54
Demodex abscesses: clinical and therapeutic challenges. Schaller M,
Sander C A, Plewig G: J Am Acad Dermatol. 2003 November; 49(5 Suppl): S272-4.
Department of Dermatology and Allergology, University of Munich, Germany.
Association between human demodicosis and HLA class I. Akilov O E,
Mumcuoglu K Y: Clin Exp Dermatol. 2003 January; 28(1): 70-3. Department of
Dermatology, Cosmetology Hospital Aesthetics, Ekaterinburg, Russian Federation.
Rosaceiform dermatitis with follicular Demodex after treatment of facial
atopic dermatitis with 1% pimecrolimus cream. Lubbe J, Stucky L, Saurat J H;
Dermatology. 2003; 207(2): 204-5
The role of bacterial antigen(s) in the etiology and persistence of
papulopustular bacteria. Dr. Kevin Kavanagh, Department of Biology, National
University of Ireland - Maynooth, and Dr. Frank Powell, consultant dermatologist,
Mater Misericordiae Hospital, Dublin. 2002. Bacteria associated with microscopic
mites known as Demodex folliculorum may play a role in the development of
papulopustular (subtype 2) rosacea, according to the results of a study funded by a
National Rosacea Society grant and reported at the 2004 annual meeting of the
Society for Investigative Dermatology.
Eucalyptus globulus (camphor oil) in the treatment of human
demodicidosis. Morsy T A, Morsy G H, Sanad E M: J Egypt Soc Parasitol. 2002
December; 32(3): 797-803. Department of Parasitology, Faculty of Medicine, Ain Shams
University, Cairo 11566, Egypt
Rosacea and the pilosebaceous follicle. Powell FC: Cutis. 2004
September; 74(3 Suppl): 9-12, 32-4. Regional Centre of Dermatology, Mater Misercordiae
Hospital, Dublin, Ireland
Demodicidosis revisited. Baima B, Sticherling M: Acta Derm Venereol.
2002; 82(1): 3-6. Department of Dermatology, University of Leipzig, Germany.
Intense pulsed light eradicates Demodex mites. (Improves Acne,
Rosacea). Timothy F. Kirn, Skin & Allergy News, Jun. 1, 2002
Increased density of Demodex folliculorum and evidence of delayed
hypersensitivity reaction in subjects with papulopustular rosacea. Georgala S,
Katoulis A C, Kylafis G D, Koumantaki-Mathioudaki E, Georgala C, Aroni K; J Eur
Acad Dermatol Venereol. 2001 September; 15(5): 441-4; National University of Athens,
Department of Dermatology and Venereology, A. Sygros' Hospital, Greece. 2001
Rosacea-like demodicidosis associated with acquired
immunodeficiency syndrome. Jansen T, Kastner U, Kreuter A, Altmeyer P: Br J
Dermatol. 2001 January; 144(1): 139-42. Department of Dermatology and Allergology,
Ruhr-University Bochum, Gudrunstrasse 56, 44791 Bochum, Germany. Full Text
Rosacea, acne and other diseases of the seborrheic spectrum. Boni R:
Schweiz Rundsch Med Prax. 2000 Mar. 30; 89(14): 566-70; Dermatologische Klinik,
UniversitatsSpital Zurich.
Rosacea, acne and other diseases of the seborrheic spectrum. Boni R:
Schweiz Rundsch Med Prax. 2000 Mar. 30; 89(14): 566-70. Dermatologische Klinik,
UniversitatsSpital Zurich.
Treatment of rosacea-like demodicidosis with oral ivermectin and
topical permethrin cream. JAAD, November 1999, part 1. Volume 41. Number 5
Christa Forstinger, MD, Harald Kittler, MD Michael Binder, MD Vienna, Austria, and
Boston, Massachusetts (Full Report if you scroll down)
Unilateral demodicidosis. Pallotta S, Cianchini G, Martelloni E, Ferranti
G, Girardelli C R, Di Leila G, Puddu P: Eur J Dermatol. 1998 April-May; 8(3): 191-2.
Department of Immunoderma-tology, Istituto Dermopatico Dell Imma-colata, IRCCS,
Via dei Monti di Creta 104, 00167 Rome, Italy.
The significance of Demodex folliculorum density in rosacea. Erbagci
Z, Ozgoztasi O: Int J Dermatol. 1998 June; 37(6): 421-5. Department of Dermatology,
Faculty of Medicine, Gaziantep University, Turkey.
Demodex-associated folliculitis. Forton F. Am J Dermatopathol. 1998
October; 20(5): 536-7
Blepharitis. Demodex folliculorum, associated pathogen spectrum and
specific therapy Demmler M; de Kaspar H M; Mohring C; Klauss V - Ophthalmologe -
1997 March; 94(3): 191-6
Pilocarpine gel for the treatment of demodicosis--a case series. Fulk
G W, Murphy B, Robins MD: Optom Vis Sci. 1996 December; 73(12): 742-5. College of
Optometry, Northeastern State University, Tahlequah, Oklahoma, USA.
Demodicidosis in childhood acute lymphoblastic leukemia; an
opportunistic infection occurring with immunosuppression. Ivy SP - Journal of
Pediatrics- 1995 November; 127(5): 751-4
Acne rosacea complicated with demodicosis. Bobrov V M; Vestn
Otorinolaringol. 1994 July-August; (4): 43-4.
Rosacea. Decauchy F, Beauvais L, Meunier L, Meynadier J: Rev Prat.
1993 Nov. 15; 43(18): 2344-8. Service de dermatologie allergologie et photobiologie,
hopital Saint-Charles, Montpellier.
The Demodex mite population in rosacea. Bonnar E, Eustace P, Powell
F C: J Am Acad Dermatol. 1993 March; 28(3): 443-8.University Department of
Ophthalmology, Mater Misercordiae Hospital, Dublin, Ireland.
Granulomatous rosacea associated with Demodex folliculorum.
Amichai B, Grunwald M H, Avinoach I, Halevy S: Int J Dermatol. 1992
October; 31(10): 718-9 Department of Dermatology, Soroka Medical Center of Kupat
Holim, Beer-Sheva, Israel.
The possible role of skin surface lipid in rosacea with epitheloid
granulomas. Basta-Juzbasic A, Marinovic T, Dobric I, Bolanca-Bumber S, Sencar J:
Acta Med Croatica. 992; 46(2): 119-23. University Department of Dermatology,
Medical Faculty, University of Zagreb, Croatia.
Topical steroid induced chronic demodicidosis. Sakuntabhai A,
Timpatanapong P: J Med Assoc Thai. 1991 February; 74(2): 116-9. Department of
Medicine, Faculty of Medicine, Ramathibodi Hospital, Mahidol University, Bangkok,
Thailand
Demodicosis, what to do about it? Vroom M W: Tijdschr Diergeneeskd.
1991 Mar. 15; 116(6): 296-7.
Papular pruritic eruption with human immunodeficiency virus infection.
Bañuls J, Ramon D, Aniz E, Jorda E, Torres V. Int J Dermatol. 1991 November;
30(11): 801-3
Unilateral demodectic rosacea. Shelley W B, Shelley E D, Burmeister V.
Department of Medicine, Medical College of Ohio, Toledo. J Am Acad Dermatol.
1989 May; 20 (5 Pt 2): 915-7.
Papulonodular demodicidosis associated with acquired
immunodeficiency syndrome.. Dominey A, Rosen T, Tschen J. J Am Acad Dermatol.
1989 February; 20(2 Pt 1): 197-201.
Demodex mites contain immunoreactive lipase. Jimenez-Acosta F,
Planas L, Penneys N. Arch Dermatol. 1989 October; 125(10): 1436-7
Rosacea: histopathologic study of 75 cases. Ramelet A A, Perroulaz G:
Ann Dermatol Venereol. 1988; 115(8): 801-6.
Service de dermatologie et de venereologie, CHUV, (Centre Hospitalier
universitaire vaudois), Lausanne, Suisse. Demodex folliculorum. Huismans H:
Klin Monatsbl Augenheilkd. 1988 September; 193(3): 304-6
Rosacea and rosacea-like demodicidosis. Ayres S: Int J Dermatol.
1987 April; 26(3): 198-9.
Demodicosis--a rosaceiform dermatosis. Wätzig V, Zollmann C:
Dermatol Monatsschr. 1987; 173(3): 158-62.
Clinical manifestations of demodicosis. Heacock C E: J Am Optom
Assoc. 1986 December; 57(12): 914-9.
T-cell subsets in acne rosacea lesions and the possible role of
Demodex folliculorum. Rufli T, Büchner S A: Dermatologica. 1984; 169(1): 1-5.
Ultra structural study of Demodex infestation of the face in healthy
subjects and acne rosacea patients : G Ital Dermatol Venereol. 1982 September-
October; 117(5): 277-81. Crosti C, Menni S, Piccinno R, Sala F.
Nosologic position of demodicidosis in humans. Bardach H G, Raff M,
Poitschek C: Hautarzt. 1981 October; 32(10): 512-8.
Demodicosis of ophthalmic concern. English F P, Nutting W B: Am J
Ophthalmol. 1981 March; 91(3): 362-72.
Demodex folliculorum: aetiopathogenesis and therapy of rosacea and
perioral dermatitis. Rufli T, Mumcuoglu Y, Cajacob A, Buchner S. Dermatologica.
1981; 162(1): 12-26.
Demodex folliculorum and rosacea: experimental and immunological
studies. Grosshans E, Dungler T, Kien T T, Kremer M: Z Hautkr. 1980 Sep.
15; 55(18): 1211-8.
Perioral dermatitis--an allergic disease? Arutjunow V: Hautarzt. 1978
February; 29(2): 89-91.
Pyroglyphid mites, xerophilic fungi and allergenic activity in dust from
hospital mattresses, v d Lustgraaf B, Jorde W: Acta Allergol. 1977 December; 32(6): 406-12.
Demodex folliculorum in rosacea. Ayers S Jr, Mihan R, Marks R,
Harcourt-Webster J N.
Demodex folliculorum and the histogenesis of granulomatous rosacea.
Grosshans E M, Kremer M, Maleville J. Hautarzt. 1974 April; 25(4): 166-77.
The role of the acarid Demodex folliculorum in ophthalmology. English
FP: Trans Aust Coll Ophthalmol. 1970; 2: 89-92.
The role of Demodex mites in the development of acne rosacea.
Kiselev O A: Vestn Dermatol Venerol. 1967 October; 41(10): 90-1.
Demodex folliculorum in patients with rosacea. Baksht B P: Vestn
Dermatol Venerol. 1966 August; 40(8): 15-22.
Demodex folliculorum and rosacea. A clinical and histological study.
Robinson T W: Arch Dermatol. 1965 November; 92(5): 542-4.
DEMODICOSIS IN MAN. AKBULATOVA LKh: Vestn Dermatol
Venerol. 1964 March; 38: 34-42.
Rosacdea-Like Demodicidosis. Samuel Ayres, Jr. Calif Med. 1963
June; 98(6): 328-330. pdf download
Demodectic eruptions (demodicidosis) in the human. 30 years'
experience with 2 commonly unrecognized entities: pityriasis folliculorum (Demodex)
and acne rosacea (Demodex type). AYRES S Jr, AYRES S 3rd: Arch Dermatol.
1961 May; 83: 816-27.
Rosacea: the role of Demodex folliculorum. BRODIE R C: Aust J
Dermatol. 1952 April; 1(3): 149-52.
Demodex folliculorum in Diseased Conditions of the Human Face.
Geo. E. Fell Proceedings of the American Society of Microscopists Published by:
Wiley-Blackwell on behalf of American Microscopical Society
Optom Vis Sci. 2013 Jun. 6. Demodex. Hom M M, Mastrota K M,
Schachter S E..
Non-invasive in vivo detection and quantification of Demodex mites by
confocal laser scanning microscopy. Sattler E C, Maier T, Hoffmann V S, Hegyi J,
Ruzicka T, Berking C. Br J Dermatol. 2012 Jun. 20. doi: 10.1111/j. 1365-
2133.2012.11096.x.
Demodex-associated Bacillus proteins induce an aberrant wound
healing response in a corneal epithelial cell line (hTCEpi). O'Reilly N, Gallagher C,
Katikireddy K, Clynes M, O'Sullivan F, Kavanagh K. Invest Ophthalmol Vis Sci. 2012
Apr. 24.
The potential role of Demodex folliculorum mites and bacteria in the
induction of rosacea. Stanislaw Jarmuda, Niamh O'Reilly, Ryszard Żaba, Oliwia
Jakubowicz, Andrzej Szkaradkiewicz and Kevin Kavanagh. Journal of Medical
Microbiology, 2012 DOI: 10.1099/jmm.0.048090-0 Article at PubMed
Facial dermatosis associated with Demodex: a case-control study.
Zhao Y E, Peng Y, Wang X L, Wu L P, Wang M, Yan H L, Xiao S X. J Zhejiang Univ Sci
B. 2011 December; 12(12): 1008-15.
Density of Demodex folliculorum in Patients Receiving Epidermal
Growth Factor Receptor Inhibitors. Gerber P A, Kukova G, Buhren B A, Homey B.
Dermatology. 2011 Feb. 22
Rosacea-like demodicidosis and chronic blepharitis. Anane S, Mokni
M, Beltaief O.. Ann Dermatol Venereol. 2011 January; 138(1): 30-34.
Demodex mites: Facts and controversies. Elston D M. Clin Dermatol.
2010 September-October; 28(5): 502-504.
Reaction to Mites May Mimic Rosacea Signs. NRS Rosacea Review,
Winter 2010, Article 6 Mites and Eye Symptoms, NRS Web Blog Thursday, Jul. 15,
2010
Criotherapy in treatment of skin demodecosis. Georgian Med News.
2009 May; (170): 43-5.
PCR analysis for Wolbachia in human and canine Demodex mites.
Borgo S N, Sattler E C, Hogardt M, Adler K, Plewig G. Department of Dermatology
and Allergology, Ludwig-Maximilian-University, Frauenlobstrasse 9-11, 80337,
Munich, Germany. Arch Dermatol Res. 2009 Aug. 4
Rejecting common wisdom: Red, scaly faces not always rosacea or
seborrheic dermatitis. Dermatology Times. Publish date: Jun. 1, 2009. By: Jane
Schwanke
Demodicosis. Manolette R Roque, M, Barbara L Roque, MD,
eMedicine, WebMD
Frequency of Demodicosis in various patient and age groups. Aycan
O M, Otlu G H, Karaman U, Daldal N, Atambay M. Inönü Üniversitesi Tip Fakültesi,
Parazitoloji Anabilim Dali, Malatya, Turkey. Turkiye Parazitol Derg. 2007; 31(2): 115-
118.
Electron microscopic investigation into the possible etiology of rosacea
and the implication for treatment, Richard Burroughs, MD, National Capital
Consortium (Walter Reed Army Medical Center), Washington, DC, United States;
Kurt Maggio, MD, Walter Reed Army Medical Center, Washington, DC, United
States., Poster Abstract P516, American Academy of Dermatology, 65th Annual
Meeting Feb. 2-6, 2007, Washington, DC. Published in Journal of the American
Academy of Dermatology Volume 56, Number 2.
Recalcitrant papulopustular rosacea in an immunocompetent patient
responding to combination therapy with oral ivermectin and topical permethrin. Allen
K J, Davis C L, Billings S D, Mousdicas N; Cutis. 2007 August; 80(2): 149-51
Clinical treatment of ocular demodecosis by lid scrub with tea tree oil.
Gao Y Y, Di Pascuale M A, Elizondo A, Tseng S C. Cornea. 2007 February; 26(2): 136-43.
Empirical treatment is key to identifying rosacea, other dermatoses.
Modern Medicine - Publish date: Nov. 1, 2007. By: John Jesitu
Demodex folliculorum and Demodex brevis as a cause of chronic
marginal blepharitis. Czepita D, Ku?na-Grygiel W, Czepita M, Grobelny A. Katedra i
Klinika Okulistyki Pomorskiej Akademii Medycznej w Szczecinie al. Powsta?ców
Wlkp. 72, 70-111 Szczecin. Ann Acad Med Stetin. 2007; 53(1): 63-7; discussion 67.
Dispelling the Mystery of Demodex. Neal Bhatia, MD and James Q Del
Rosso, DO, FAOCD US PHARMACY REVIEW 2006, p. 38-41
Could matrix metalloproteinase-9 be a link between Demodex
folliculorum and rosacea? R R Bonamigo, L Bakos, M Edelweiss, A Cartell. Journal of
the European Academy of Dermatology & Venereology. Volume 19 Page 646 -
September 2005, Volume 19 Issue 5
A clinico-pathological approach to the classification of human
demodicosis. Akilov O E, Butov Y S, Mumcuoglu K Y: J Dtsch Dermatol Ges. 2005
August; 3(8): 607-14. Department of Dermatology, Cosmetology Hospital “Aesthetics”,
Ekaterinburg, Russian Federation.
The role of HLA A2 and Cw2 in the pathogenesis of human
demodicosis. Mumcuoglu K Y, Akilov O E: Dermatology. 2005; 210(2): 109-14.
Department of Parasitology, Hebrew University-Hadassah Medical School,
Jerusalem, Israel.
Assen L. Dourmishev, Lyubomir A. Dourmishev, Robert A. Schwartz
(2005) Ivermectin: pharmacology and application in dermatology. International
Journal of Dermatology 44 (12), 981-988. doi: 10.1111/j.1365-4632.2004.02253.x
Topical application of 1-methylnicotinamide in the treatment of rosacea:
a pilot study. Wozniacka A, Wieczorkowska M, Gebicki J, Sysa-Jedrzejowska A: Clin
Exp Dermatol. 2005 November; 30(6): 632-5. Department of Dermatology, Medical
University of Lodz, Poland.
Rosacea and Demodex. Rufli T: J Dtsch Dermatol Ges. 2005 August; 3
(8): 585-6.
[Investigations on the occurrence as well as the role of Demodex
follicuforum and Demodex brevis in the pathogensis of blepharitis], Czepita D,
Ku?na-Grygiel W, Kosik-Bogacka D. Klin Oczna. 2005; 107(1-3): 80-2
Consider Demodex mites regardless of immune status. Skin; Allergy
News, September, 2005 by Patrice Wendling
Rosacea: I. Etiology, pathogenesis, and subtype classification.
Crawford G H, Pelle M T, James W D. Department of Dermatology, University of
Pennsylvania Medical Center, USA: J Am Acad Dermatol. 2004 September; 51(3): 327-41;
quiz 342-4.
Rosacea and the pilosebaceous follicle. Powell F C: Cutis. 2004
September; 74(3 Suppl): 9-12, 32-4. Regional Centre of Dermatology, Mater Misercordiae
Hospital, Dublin, Ireland.
The clinical importance of Demodex folliculorum presenting with
nonspecific facial signs and symptoms. Karincaoglu Y, Bayram N, Aycan O,
Esrefoglu M: J Dermatol. 2004 August; 31(8): 618-26.
Efficiency of benzoyl peroxide-erythromycin gel in comparison with
metronidazole gel in the treatment of acne rosacea. Ozturkcan S, Ermertcan AT,
Sahin M T, Afsar F S; J Dermatol. 2004 August; 31(8): 610-7; Department of Dermatology,
Medical Faculty of Celal Bayar University, Manisa, Turkiye.
Facial demodicosis. Zomorodian K, Geramishoar M, Saadat F,
Tarazoie B, Norouzi M, Rezaie S; Eur J Dermatol. 2004 March-April; 14(2): 121-2. Div. of
Molecular Biology, Dept, of Medical Mycology & Parasitology, School of Public
Health and Institute of Public Health Research, Tehran University of Medical
Sciences, P.O. Box 14155, 64410 Tehran, Iran.
Treatment of human Demodex folliculorum by camphor oil and
metronidazole. El-Shazly A M, Hassan A A, Soliman M, Morsy G H, Morsy T A; J Egypt
Soc Parasitol. 2004 April; 34(1): 107-16. Departments of Parasitology, Faculty of
Medicine, Mansoura University, Mansoura, Egypt.
Induction of rosaceiform dermatitis during treatment of facial
inflammatory dermatoses with tacrolimus ointment. Antille C, Saurat J H, Lubbe J;
Arch Dermatol. 2004 April; 140(4): 457-60. Department of Dermatology, University
Hospital, Geneva, Switzerland.
It's Enough To Make Your Skin Crawl: Microscopic Mites May Be
Linked To Acne, Thinning Hair And Other Skin Disorders. Chuck Woods:
04.23.2003. University of Florida's Institute of Food and Agricultural Sciences
Is permethrin 5% cream effective for rosacea? Swenor M E: J Fam
Pract. 2003 March; 52(3): 183-4. Harrisburg Family Practice, Residency Program,
PinnacleHealth Hospitals, Pa, USA
Symbiotic intraceullular bacteria of Demodex folliculorum and the
pathogenesis of rosacea. Drs. Richard Burroughs, Mark Peake and Richard Vinson,
of William Beaumont Army Medical Center; Dr. Scott Norton, chief, Dermatology
Service, Walter Reed Army Medical Center; and Dr. John Werren, professor of
biology, and Seth Bordenstein, University of Rochester.
The researchers were awarded $12,250 to test Demodex from rosacea
patients for the presence of bacteria, and analyze data for a possible statistical or
clinical link between the bacteria and the presence of rosacea. They hypothesize
that the cutaneous changes of rosacea may be due to an inflammatory response to
bacteria within Demodex rather than the mite itself. Status: Interim report submitted
April 2003. Study continues [editor's note: what happened to this report? Why pay
this and wait this long for a final report?]
Effects of intense pulsed light on sun-damaged human skin, routine,
and ultrastructural analysis. Prieto V G, Sadick N S, Lloreta J, Nicholson J, Shea C R:
Lasers Surg Med. 2002; 30(2): 82. Department of Pathology, UT-MD Anderson
Cancer Center, Houston, Texas 77030, USA.
The management of rosacea. Rebora A: Am J Clin Dermatol.
2002; 3(7): 489-96. Department of Endocrinological and Metabolic Diseases, Section
of Dermatology, University of Genoa, Genoa, Italy.
Permethrin 5% cream versus metronidazole 0.75% gel for the
treatment of papulopustular rosacea. A randomized double-blind placebo-controlled
study. Kocak M, Yagli S, Vahapoglu G, Eksioglu M: Dermatology. 2002; 205(3): 265-
70. Department of Dermatology, Kirikkale University, Faculty of Medicine, Kirikkale,
Turkey.
Demodex folliculorum in development of dermatitis rosaceiformis
steroidica and rosacea-related diseases. Basta-Juzbasi? A, Subi? J S, Ljubojevi? S.
Clin Dermatol. 2002 March-April; 20(2): 135-40..
The pathogenesis of Demodex folliculorum (hair follicular mites) in
females with and without rosacea. el-Shazly A M, Ghaneum B M, Morsy T A, Aaty H E:
J Egypt Soc Parasitol. 2001 December; 31(3): 867-75. Department of Parasitology, Faculty
of Medicine, Mansoura University, Egypt.
Is Demodex really non-pathogenic? Pena G P, Andrade Filho J S: Rev
Inst Med Trop Sao Paulo. 2000 May-June; 42(3): 171-3. Laboratorio Distrital Centro-
Sul, Prefeitura de Belo Horizonte, Minas Gerais, Brasil
Tubero-pustular demodicosis. Grossmann B, Jung K, Linse R.
Hautarzt. 1999 July; 50(7): 491-4.
Limitations of standardized skin surface biopsy in measurement of the
density of Demodex folliculorum. A case report. Forton F, Song M: Br J Dermatol.
1998 October; 139(4): 697-700. Clinic of Dermatology, Universite Libre de Bruxelles, Saint
Pierre University Hospital, Brussels, Belgium
Demodex mites in acne rosacea. Roihu T, Kariniemi A L: J Cutan
Pathol. 1998 November; 25(10): 550-2. Department of Dermatology, Helsinki University
Central Hospital, Finland.
Demodex folliculorum and topical treatment: acaricidal action evaluated
by standardized skin surface biopsy. Forton F, Seys B, Marchal J L, Song A M. Br J
Dermatol. 1998 March; 138(3): 461-6.
A study on Demodex folliculorum in rosacea. Abd-El-Al A M, Bayoumy
A M, Abou Salem E A: J Egypt Soc Parasitol. 1997 April; 27(1): 183-95. Department of
Dermatology, Faculty of Medicine, Al-Azhar University, Nasr City, Cairo
Rosacea-like demodicosis in an HIV-positive child. Barrio J, Lecona M,
Hernanz J M, Sanchez M, Gurbindo M D, Lazaro P, Barrio J L: Dermatology.
1996; 192(2): 143-5. Dermatology, Service, Hospital General Universitario Gregorio
Maranon, Madrid, Spain.
Demodicidosis or rosacea: what did we treat? Hoekzema R,
Hulsebosch H J, Bos J D: Br J Dermatol. 1995 August; 133(2): 294-9. Department of
Dermatology, Academisch Medisch Centrum, University of Amsterdam, The
Netherlands.
Demodex mites in rosacea. Diaz-Perez J L: J Am Acad Dermatol. 1994
May; 30(5 Pt 1): 812-3.
Demodex-attributed rosacea-like lesions in AIDS. Redondo Mateo J,
Soto Guzmán O, Fernandez Rubio E, Domínguez Franjo F: Acta Derm Venereol.
1993 December; 73(6): 437.
Density of Demodex folliculorum in rosacea: a case-control study using
standardized skin-surface biopsy. Forton F, Seys B. Br J Dermatol 1993; 128: 650-9.
Rosacea: a study of clinical patterns, blood flow, and the role of
Demodex folliculorum. Sibenge S, Gawkrodger D J: J Am Acad Dermatol. 1992
April; 26(4): 590-3. Department of Dermatology, University of Sheffield, Royal
Hallamshire Hospital, U.K.
Demodicidosis in a child with leukemia. Sahn E E, Sheridan D M. J Am
Acad Dermatol. 1992 November; 27(5 Pt 2): 799-801.
Demodicosis and rosacea. Skrlin J, Richter B, Basta-Juzbasic A,
Matica B, Ivacic B, Cvrlje M, Kucisec N, Baucic A: 1991 Mar. 23; 337(8743): 734.
[Demodex folliculorum and Demodex brevis (Acarida) as the factors of
chronic marginal blepharitis] Humiczewska M. Wiad Parazytol. 1991; 37(1): 127-30.
Papular pruritic eruption of Demodex folliculitis in patients with acquired
immunodeficiency syndrome. Ashack R J, Frost M L, Norins A L. J Am Acad Dermatol.
1989 August; 21(2 Pt 1): 306--7
Pityriasis folliculorum revisited. Dominey A, Tschen J, Rosen T, Batres
E, Stern J K. J Am Acad Dermatol. 1989 July; 21(1): 81-4.
The hair follicle mites (Demodex spp.). Could they be vectors of
pathogenic microorganisms? Wolf R, Ophir J, Avigad J, Lengy J, Krakowski A: Acta
Derm Venereol. 1988; 68(6): 535-7. Department of Dermatology, Ichilov Medical
Center, Tel-Aviv, Israel.
Demodicidosis mimicking granulomatous rosacea and transient
acantholytic dermatosis (Grover's disease). Lindmaier A, Jurecka W, Lindemayr H:
Dermatologica. 1987; 175(4): 200-4.
Demodex and perifollicular inflammation in man: review and report of
69 biopsies. Forton F. Ann Dermatol Venereol. 1986; 113(11): 1047-58
Rosacea and demodicosis (a review of the literature) Glukhen'ki? B T,
Snitsanenko O V. Vrachebnoe Delo. 1984 February; (2): 94-6.
The hair follicle mites Demodex folliculorum and Demodex brevis:
biology and medical importance. A review. Rufli T, Mumcuoglu Y. Dermatologica.
1981; 162(1): 1-11.
Metronidazole and Demodex folliculorum. Persi A, Rebora A: Acta
Derm Venereol. 1981; 61 (2): 182-3
Pathogenesis associated with hair follicle mites (Demodex spp.) in Australian
Aborigines. Nutting W B, Green A C: Br J Dermatol. 1976 March; 94(3): 307-12.
Demodecidosis and rosaceiform dermatitis. Hojyo Tomoka M T,
Dominguez Soto L: Med Cutan Ibero Lat Am. 1976; 4(2): 83-90.
Rosacea-like demodicidosis involving the eyelids. A case report. Ayres
S, Mihan R: Arch Dermatol. 1967 January; 95(1): 63-6.
The pathogenic role of the mite Demodex and the clinical forms of demodicosis
in man. Akbulatova LKh: Vestn Dermatol Venerol. 1966 December; 40(12): 57-61.
U.S. patent application Pub. No. 2011/0033395; U.S. Pat. Nos. 5,952,372
and 5,629,300; PCT Pub. WO 2009/010754; EP Pub. EP2355790.

I claim:

1. A method of treating a skin affliction, the method comprising the step of administering to the individual chloroquine and/or hydroxychloroquine in a dosage sufficient to inactivate at least a portion of Demodex mites in the human body, wherein the inactivation results in attenuation or cessation of one or more symptoms associated with the skin affliction.

2. The method of claim 1, wherein substantially all Demodex mites in the human body are inactivated.

3. The method of claim 1, wherein the one or more clinical symptom is associated with an allergic and/or vasomotor response to the Demodex mites that cause the skin affliction or symptoms thereof.

4. The method of claim 1, wherein the chloroquine and/or hydroxychloroquine is provided at said dosage sufficient to kill said Demodex mites from hair follicles and/or skin of the individual contacted with said chloroquine and/or hydroxychloroquine.

5. The method of claim 1, wherein said dosage is sufficient to provide a reduction in the population of said Demodex mites from said hair follicles or skin contacted with said chloroquine and/or hydroxychloroquine that is greater than or equal to 80%.

6. The method of claim 5, wherein said reduction in the population is by one or more of: killing adult mites; killing mite larva; or affecting a fertilized egg to prevent emergence of a viable larva.

7. The method of claim 5, wherein said dosage is sufficient to provide for said reduction in the population of said Demodex mites over a time interval that is up to 1 month.

8. The method of claim 5, further comprising re-applying or re-administering said chloroquine and/or hydroxychloroquine in a dosage sufficient to maintain said reduction in said population of said Demodex mites over a time interval greater than or equal to two months.

9. The method of claim 8, wherein the re-applying or re-administering is synchronized with a life cycle of the mites so as to inactivate a second generation of mites that were in an egg or larval form during the initial orally administering or topically applying step and before a substantial portion of the second generation of mites are capable of reproducing.

10. The method of claim 9, further comprising re-applying or re-administering a second time, wherein the time interval between temporally adjacent applications is independently selected over a time that is greater than or equal to 3 days and less than or equal to 7 days.

11. The method of claim 1, wherein said dosage is sufficient to render said Demodex mites incapable of reproducing in or on hair follicles and/or skin of the individual contacted with said chloroquine and/or hydroxychloroquine.

12. The method of claim 1, wherein said chloroquine and/or hydroxychloroquine is provided at said dosage sufficient to fill pores of said skin, hair follicles or both of the individual contacted with said chloroquine and/or hydroxychloroquine.

13. The method of claim 1, wherein the skin affliction affects facial skin or eyelids, or both.

14. The method of claim 1, wherein the skin affliction is one or more of common acne, seborrheic dermatitis, perioral dermatitis, atopic dermatitis, an acneform rash, transient acantholytic dermatosis, acne necrotica milliaris, psoriasis, steroid induced dermatitis, primary irritation dermatitis or rosacea.

15. The method of claim 1, wherein the skin affliction is rosacea.

16. The method of claim 1, wherein the skin affliction is erythematotelangiectatic rosacea, papulopustular rosacea, phymatous rosacea, ocular rosacea or rhinophyma.

17. The method of claim 1, wherein the skin affliction is acne vulgaris.

18. The method of claim 1, wherein said chloroquine and/or hydroxychloroquine is a miticide or an insecticide.

19. The method of claim 18, wherein said chloroquine and/or hydroxychloroquine kills Demodex brevis mites, Demodex folliculorum mites or both.

20. The method of claim 18, wherein said chloroquine and/or hydroxychloroquine kills larva or eggs of said Demodex brevis mites, Demodex folliculorum mites or both.

21. The method of claim 1, wherein said chloroquine and/or hydroxychloroquine is transported into an epidermis or a subdermal region upon contact with said hair follicles and/or skin of the individual.

22. The method of claim 21, wherein said chloroquine and/or hydroxychloroquine is characterized by a transport rate into said epidermis or a subdermal region upon contact with said hair follicles and/or skin of the individual equal to or greater than 0.1 microns per second.

23. The method of claim 1, wherein the chloroquine and/or hydroxychloroquine is characterized by a biological half-life less than or equal to 15 minutes.

24. The method of claim 1, wherein the chloroquine and/or hydroxychloroquine topically applied as formulated in a carrier lotion, cream, soap, wash, shampoo or gel.

25. The method of claim 24, wherein the chloroquine and/or hydroxychloroquine has a concentration in the topically applied carrier lotion, cream, soap, wash, shampoo or gel selected from the range 0.001 to 5 percent by weight.

26. The method of claim 24, wherein the chloroquine and/or hydroxychloroquine in the topically applied carrier lotion, cream, soap, wash, shampoo or gel is provided in a lowest concentration effective for killing the Demodex brevis mites, Demodex folliculorum mites or both.

27. The method of claim 24, comprising providing a dosage of said chloroquine and/or hydroxychloroquine in the topically applied carrier lotion, cream, soap, wash, shampoo or gel less than 150 mg/kg of body mass.

28. The method of claim 24, comprising providing a dosage of said chloroquine and/or hydroxychloroquine in the topically applied carrier lotion, cream, soap, wash, shampoo or gel selected from the range of 0.01 mg per kg of body mass to 50 mg/kg of body mass.

29. The method claim 24, wherein said dosage of chloroquine and/or hydroxychloroquine in the topically applied carrier lotion, cream, soap, wash, shampoo or gel is a lowest dose effective for killing the Demodex mites.

30. The method of claim 24, wherein the topically applied chloroquine and/or hydroxychloroquine is encapsulated inside microliposomes or micelles.

31. The method of claim 24, wherein the topically applied chloroquine and/or hydroxychloroquine is provided as an emulsion in said topically applied lotion, cream, soap, wash, shampoo or gel.

32. The method of claim 1, comprising topically applying said chloroquine and/or hydroxychloroquine to skin areas exhibiting the clinical symptom.

33. The method of claim 32, further comprising topically applying said chloroquine and/or hydroxychloroquine to skin areas not exhibiting the clinical symptom.

34. The method of claim 1, further comprising topically applying said chloroquine and/or hydroxychloroquine to skin and hair areas of the body where Demodex brevis mite or Demodex folliculorum mites are present.

35. The method of claim 1, further comprising topically applying said chloroquine and/or hydroxychloroquine to all skin areas of said individual.

36. The method of claim 1, further comprising topically applying said chloroquine and/or hydroxychloroquine to all hair areas of said individual.

37. The method of claim 1, further comprising a step of applying the chloroquine and/or hydroxychloroquine to the individual's clothing, linens or both clothing and linens.

38. The method of claim 1, further comprising a step of orally administering or topically applying the chloroquine and/or hydroxychloroquine to others having contact with the individual in a dosage sufficient to inactivate Demodex brevis mites, Demodex folliculorum mites or both from hair follicles or skin of the others.

39. The method of claim 38, wherein the others comprise household members, children, spouses, partners, family members or pets.

40. The method of claim 1, wherein the topically applied chloroquine and/or hydroxychloroquine is applied to the hair follicles and skin of the individual.

41. The method of any of claim 1, wherein the topically applied chloroquine and/or hydroxychloroquine penetrates an outer layer of the skin of the individual, thereby exposing the Demodex brevis mites, Demodex folliculorum mites or both present below the outer layer of the skin to the chloroquine and/or hydroxychloroquine.

42. The method of claim 1, wherein the topically applied chloroquine and/or hydroxychloroquine penetrates to a subdermal region of the skin of the individual, thereby exposing the Demodex brevis mites, Demodex folliculorum mites or both present in the subdermal region of the skin to the chloroquine and/or hydroxychloroquine.

43. The method of claim 1, wherein the topically applied chloroquine and/or hydroxychloroquine is applied to affected skin areas at least once and not more than twice daily for a period of two to twelve weeks.

44. The method of claim 1, wherein the topically applied chloroquine and/or hydroxychloroquine is applied to the affected skin areas and/or to non-affected skin areas during a first application period, thereby inactivating said Demodex brevis mites, Demodex folliculorum mites or both from the hair follicles in the skin of the individual.

45. The method of claim 44, wherein the topically applied chloroquine and/or hydroxychloroquine is further applied to the affected skin areas and/or to non-affected skin areas during a second application period, thereby inactivating said Demodex brevis mites, Demodex folliculorum mites or both from the hair follicles and/or skin of the individual that have matured from a larval form and/or an egg form present on and/or in the skin during or after the first application period.

46. The method of claim 45, wherein the topically applied chloroquine and/or hydroxychloroquine is further applied to the affected skin areas and/or to non-affected skin areas during a third application period, thereby inactivating said Demodex brevis mites, Demodex folliculorum mites or both from the hair follicles and or skin of the individual Demodex brevis and/or Demodex folliculorum mites that have matured from a larval form and/or an egg form present on and/or in the skin and/or the hair follicles during or after the first application period and/or the second application period.

47. The method of claim 45, wherein the first application period and the second application period are separated by at least three days and not more than seven days.

48. The method of claim 45, wherein the first application period and the second application period are separated by at least three days.

49. The method of claim 45, wherein the first application period and the second application period are separated by a time sufficient to allow larva of said Demodex brevis mites, Demodex folliculorum mites or both to mature into an adult form and/or to allow eggs of said Demodex brevis mites, Demodex folliculorum mites or both to mature into the adult form.

50. The method of claim 46, wherein the second application period and the third application period are separated by at least three days and not more than seven days.

51. The method of claim 46, wherein the second application period and the third application period are separated by at least three days.

52. The method claim 46, wherein the second application period and the third application period are separated by a time sufficient to allow time sufficient to allow larva of said Demodex brevis mites, Demodex folliculorum mites or both to mature into an adult form and/or to allow eggs of said Demodex brevis mites, Demodex folliculorum mites or both to mature into the adult form.

53. The method of claim 1, wherein the chloroquine and/or hydroxychloroquine is orally administered or topically applied in a continued intermittent regime sufficient for prophylactic control of Demodex mite population in the hair follicles and/or skin of the individual.

54. The method of claim 1, wherein the administering is by oral administration at an oral dose equal to or less than 150 mg per kg of body mass.

55. The method of claim 54, wherein the oral dose is selected from the range of 0.01 mg per kg of body mass and 50 mg per kg of body mass.

56. The method of claim 1, wherein the chloroquine and/or hydroxychloroquine is orally administered at an oral dose that is a lowest dose effective for killing the Demodex mites.

57. The method of claim 54, wherein the orally administered chloroquine and/or hydroxychloroquine is administered as a daily dose of 1 to 20 mg per kg of body mass, optionally 10 mg per kg of body mass.

58. The method of claim 54, wherein the orally administered chloroquine and/or hydroxychloroquine is administered as a daily dose of 1 to 10 mg per kg of body mass, optionally 7.5 mg per kg of body mass.

59. The method of claim 54, wherein the orally administered chloroquine and/or hydroxychloroquine is administered as a three times per day dose of 1 to 10 mg per kg of body mass, optionally 5 mg per kg of body mass.

60. The method of claim 54, wherein the orally administered chloroquine and/or hydroxychloroquine is repeated two to four times with spacing of three to seven days between them.

61. The method of claim 54, wherein the orally administered chloroquine and/or hydroxychloroquine is formulated as a prodrug or pharmaceutically acceptable salt or ester.

62. The method of claim 1, wherein the inactivation of the Demodex brevis and/or Demodex folliculorum mites from hair follicles and/or skin of the individual results in a reduction in population of one or more bacteria in the hair follicles and/or skin of the individual.

63. The method of claim 1, wherein the manifestations of allergic and/or vasomotor responses to the mites result from a presence of one or more bacteria associated with the mites in the hair follicles and/or skin of the individual.

64. The method of claim 62, wherein the one or more bacteria comprise one or more bacteria from the genus staphylococcus or from the genus bacillus.

65. The method of claim 62, wherein the one or more bacteria comprise bacillus oleronius.

66. The method of claim 62, wherein the one or more bacteria comprise Staphylococcus epidermidis.

67. The method of any of claim 62, wherein the one or more bacteria are present in a digestive system of the Demodex brevis and/or Demodex folliculorum mites.

68. A method of treating a skin affliction comprising a step of topically applying an active ingredient in a dosage to an individual having the skin affliction to inactivate Demodex brevis mites, Demodex folliculorum mites or both from hair follicles or skin of said individual, resulting in attenuation or cessation of a clinical symptom associated with the skin affliction, wherein the topically applied active ingredient is applied to skin areas exhibiting the clinical symptom and to skin areas not exhibiting the clinical symptom.

69. The method of claim 68, wherein the clinical symptom is a manifestation of allergic and/or vasomotor responses to the Demodex brevis mites, Demodex folliculorum mites or both Demodex brevis mites and Demodex folliculorum mites.

70. The method of claim 68, wherein the topically applied active ingredient is applied to all skin of the individual to inactivate Demodex brevis mites, Demodex folliculorum mites or both from all skin of the individual.

71. The method of claim 68, wherein the active ingredient is formulated in a shampoo or body-wash and the shampoo or body-wash is applied to at least 50% of the total surface area of skin of the individual.

72. The method of claim 68, wherein the active ingredient comprises hydroxychloroquine.

73. The method of claim 68, wherein said chloroquine and/or hydroxychloroquine kills Demodex brevis mites, Demodex folliculorum mites or both.

74. The method of claim 68, wherein said chloroquine and/or hydroxychloroquine kills larva or eggs of said Demodex brevis mites, Demodex folliculorum mites or both.

75. The method of claim 68, wherein the skin affliction comprises one or more of common acne, atopic dermatitis, seborrheic dermatitis, perioral dermatitis, an acneform rash, transient acantholytic dermatosis, acne necrotica milliaris, psoriasis, steroid induced dermatitis, primary irritation dermatitis or rosacea.

76. The method of claim 1 or 68, further comprising co-administration or post-administration with intense light or intense pulsed light.

77. The method of claim 1 or 68, wherein the method of treating is for a human patient.

78. A method for determining presence or absence of a skin affliction caused by Demodex brevis mites, Demodex folliculorum mites, or both in a patient, the method comprising the steps of:
applying to a skin application region of the patient a chloroquine and/or hydroxychloroquine having an inactivation activity for Demodex brevis mites, Demodex folliculorum mites, or both for an application time; and
observing the skin application region for a flare-up response;
wherein an observable flare-up response indicates presence of the skin affliction caused by Demodex brevis mites, Demodex folliculorum mites, or both; and
a no observable flare-up response indicates absence of the skin affliction caused by Demodex brevis mites, Demodex folliculorum mites, or both.

79. The method of claim 78, further comprising the step of, for a patient having the observable flare-up response, treating the patient by:
topically applying to the patient the chloroquine and/or hydroxychloroquine in a dosage sufficient to inactivate mites from hair follicles or skin of said individual, resulting in attenuation or cessation of a one or more clinical symptom of the skin affliction.

80. The method of claim 79, wherein the application time is selected from a range that is greater than or equal to 10 seconds and less than or equal to 2 minutes.

81. The method of claim 79, wherein the application region has a surface area that is less than or equal to 10 cm².

82. The method of claim 79, wherein the applying step comprises applying a substrate layer having a contact face that supports the chloroquine and/or hydroxychloroquine to the skin application region.

83. The method of claim 79, wherein the flare-up response comprises an immune response and/or a vasomotor response.

84. The method of claim 82, wherein the flare-up response is one or more of localized reddening, discoloration, swelling, itching, or irritation.

85. A method of alleviating in a subject symptoms associated with an autoimmune disease caused by Demodex organisms, the method comprising administering chloroquine and/or hydroxychloroquine to the subject having the autoimmune disease in a dosage sufficient to inactivate at least a portion of the Demodex organisms.

86. The method of claim 85, further comprising the step of identifying a subject in need of alleviating symptoms associated with the autoimmune disease caused by demodex organisms.

87. The method of claim 85 or 86, further comprising the step of monitoring whether the subject experiences symptom alleviations.

88. The method of any of claims 85-87, wherein the administration is topically, orally, or intravenously providing the chloroquine and/or hydroxychloroquine to the subject.

89. The method of any of claims 85-88, wherein the administration is a topical administration.

90. The method of claim 89, wherein the topical administration comprises applying the chloroquine and/or hydroxychloroquine to the face and hair follicles of the face and head.

91. The method of claim 89, wherein the topical administration comprises applying the chloroquine and/or hydroxychloroquine to substantially the entire body.

92. The method of any of claims 85-91, further comprising the step of sampling demodex levels in the patient after the administering step.

93. The method of claim 92, wherein the sampling step comprises one or more of visualization of a skin surface, swabbing a skin surface, removing hair, a skin surface biopsy using an adhesive, a skin biopsy, or staining to visualize demodex such as by Löffler's alkaline methylene blue staining.

94. The method of claim 24, wherein the chloroquine and/or hydroxychloroquine has a concentration in the topically applied carrier lotion, cream, soap, wash, shampoo or gel of between 0.001 percent to 15 percent by weight.
